(19) United States
(12) Patent Application Publication
(10) Pub. No.: US 2024/0293308 A1
(43) Pub. Date: Sep. 5, 2024

(54) IMPLANTABLE CELL MACROENCAPSULATION DEVICE AND METHOD OF MANUFACTURE AND USE

(71) Applicant: The Brigham and Women's Hospital, Inc., Boston, MA (US)

(72) Inventors: Jeffrey M Karp, Brookline, MA (US); Kisuk Yang, Cambridge, MA (US); Allison E Hamilos, Brookline, MA (US); Eoin O'Cearbhaill, Boston, MA (US); Peter Anthony Jones, Medford, MA (US)

(21) Appl. No.: 17/915,606

(22) PCT Filed: Apr. 2, 2021
(86) PCT No.: PCT/US21/25545

§ 371 Date: Sep. 29, 2022

Related U.S. Application Data
(60) Provisional application No. 63/004,841, filed on Apr. 3, 2020.

(51) Int. Cl. A61K 9/00; A61F 2/02; A61K 35/39; B33Y 10/00; B33Y 80/00

(52) U.S. Cl. CPC A61K 9/0024; A61F 2/022; A61K 35/39; B33Y 10/00; B33Y 80/00

(57) ABSTRACT

A cell encapsulating implantable device, including: a cell chamber accommodating a plurality of biological cells disposed within a fluid, the cell chamber at least partially enclosed within an immuno-isolative membrane which permits diffusive exchange of nutrients between the fluid and a tissue in which the device is implanted for sustaining the plurality of biological cells, and the cell chamber being configured to accommodate flow of fluid therethrough.

### CROSS-REFERENCE TO RELATED APPLICATIONS

This application is a 371 application of PCT/US2021/025545 filed Apr. 2, 2021, which claims priority from U.S. Patent Application Ser. No. 63/004,841, filed on Apr. 3, 2020, the entire disclosure of which is incorporated herein by reference.

### STATEMENT REGARDING FEDERALLY SPONSORED RESEARCH

This invention was made with government support under grant numbers HL095722 and U01DK104218 awarded by the National Institutes of Health. The government has certain rights in the invention.

### BACKGROUND

Current Type 1 diabetes treatments rely primarily on exogenous insulin replenishment via local injection. An alternative approach, namely whole pancreas or ß-cell islet transplantation, either allogeneic or xenogeneic, has the potential advantage of sustained insulin production to restore normoglycemia, but cell sources are scarce and require life-long immunosuppressants, which exhibit long term side effects. To avoid immunosuppression, encapsulation approaches have focused on achieving immune-isolation of transplanted cells. However, macroencapsulation approaches to date have not been completely satisfactory.

### SUMMARY

Thus, what are need are improved apparatus and methods for macroencapsulation of implanted cells. Accordingly, the methods and systems disclosed herein provide improved apparatus and methods for macroencapsulation of implanted cells which provide for convective flow of perfusate in an immuno-isolative manner.

In one embodiment, the disclosure provides a cell encapsulating implantable device, including: a cell chamber accommodating a plurality of biological cells disposed within a fluid, the cell chamber at least partially enclosed within an immuno-isolative membrane which permits diffusive exchange of nutrients between the fluid and a tissue in which the device is implanted for sustaining the plurality of biological cells, and the cell chamber being configured to accommodate flow of fluid therethrough.

In another embodiment, the disclosure provides a method of fabricating a cell encapsulating implantable device, the device including: a cell chamber accommodating a plurality of biological cells disposed within a fluid, the cell chamber at least partially enclosed within an immuno-isolative membrane which permits diffusive exchange of nutrients between the fluid and a tissue in which the device is implanted for sustaining the plurality of biological cells, the method including: additively applying 3D printing materials; applying the immuno-isolative membrane to the 3D materials by conformal spray coating; and generating a hollow fiber which extends through the cell chamber, the hollow fiber including a semi-permeable surface in communication with the plurality of biological cells.

In yet another embodiment, the disclosure provides a method of providing therapy for a disease or condition, including: fabricating a cell encapsulating implantable device for encapsulating biological cells that treat the disease or condition; and implanting the device in a patient exhibiting the disease or condition, wherein the plurality of biological cells are loaded into the device pre-implantation or post-implantation.

In still another embodiment, the disclosure provides a cell encapsulating implantable device, including: a cell chamber accommodating a plurality of biological cells disposed within a fluid, the cell chamber at least partially enclosed within an immuno-isolative membrane which permits diffusive exchange of nutrients between the fluid and a tissue in which the device is implanted for sustaining the plurality of biological cells, and the cell chamber being provided as a condensed 3D shape.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows a design of a convection-enhanced macroencapsulation device (ceMED) for increasing mass transport, B-cell viability, and insulin secretion sensitivity. Panel (A) provides an Illustration comparing diffusion-based versus convection-enhanced approaches. Expanding macroencapsulation systems, from typical 2D wafer static systems, brings forth mass transport limitations and cell death. These limitations motivate the introduction of a hollow fiber (HF) in an expanded 3D MED to allow increased nutrient delivery by perfused flow in the ceMED. Panel (B) provides a simulation showing the gradient of oxygen (mM), glucose concentration (mM), and insulin secretion rate (nM/s) as a function of position inside a static macroencapsulation with multiple layers of islets. The color bars indicate the concentration of each variables. White arrows indicate the hypoxic regions in the islet due to diffusion limited transport of the oxygen in the device. Panel (C) shows a scheme of ceMED which includes an equilibrium chamber (EqC) and a cell chamber (CC) connected by a HF. EqC captures glucose and oxygen from surroundings to be transported to encapsulated cells in the CC. Inside the CC, positive pressure facilitates flow and improved mass transport to and from the encapsulated cells. The CC is enclosed by a polytetrafluoroethylene (PTFE) membrane for protection from immune attack while allowing nutrient transfer. Panel (D) shows a gross view of fully assembled implantable ceMED and its components, which can be connected to a variety of pump systems exemplified here by an osmotic pump.

FIG. 2 shows in vitro optimization of the ceMED shows that lower flow rates and longer HF allows improved glucose equilibration. Panel (A) shows modeling of oxygen, glucose, and insulin transport in static and ceMED using COMSOL®. The snapshot is obtained after 7 min. Panel (B) shows an illustration of an experimental setup for equilibrium of EqC in panels (C) and (D). The EqCs are submerged in 5 mM glucose solution and pumped with PBS through the HF. Outflow glucose concentration profiles are measured by Amplex red assay when changing either the flow rates ejected by the syringe pump (100, 500, and 1,000 μl/h) or the lengths of EqC (5, 10, and 20 mm). Panel (C) shows that a lower flow rate allows longer residence time and improved equilibration (n=3). Panel (D) shows that a longer HF length leads to more surface area and improved equilibration as measured by glucose outflow concentration. Data are compiled to show the efficiency of three HF lengths (5, 10, and 20 mm) at 100 μl/h flow rate (n=3). Panel (E) shows an illustration of an experimental setup for panels (F) and (G). Devices are submerged in glucose solution and pumped with PBS through the HF. Glucose concentration profile in the CC (with 10 mm HF) is directly collected and measured. Panel (F) shows flow groups which show increased glucose equilibration in CC compared to no flow group (n=3). Panel (G) shows monitoring the response of changes in CC glucose by mimicking a surrounding environment with dynamic glucose changes (5 mM to 13 mM to 5 mM, respectively) (n=3).

FIG. 3 depicts how ceMED shows increased viability and insulin secretion sensitivity of encapsulated cells affording a higher loading capacity. Panels (A) and (B) indicate that ceMEDs show less cell death as seen by TUNEL analysis at the outer and middle section of the device. Lower quantification of apoptotic MIN6 cells is seen in flow groups compared to no flow group after 3 days in culture (n=4, *** P<0.001 versus no flow group). N.S., not significant. Panel (C) shows sequential concentration of insulin secreted from MIN6 cells cultured in the devices show a faster on/off rate of secretion in flow group in glucose-stimulated insulin secretion (GSIS) test (n=4). Panel (D) shows cumulative GSIS indices from the MIN6 cells taken at 15 and 30 minutes are increased in the flow group than no flow group. The dashed lines indicate the GSIS index range for static MIN6 cells (n=4, *** P<0.001 versus no flow group). Panel (E) shows gross morphology of stem cell derived ß clusters (SCBCs) in the CC. Panels (F) and (G) show optimization of loading capacity of SCBCs in the device (2.5˜20 IEQ/μl). Perfused flow allows loading of higher cell density than no flow group (n=3, *P<0.05, versus no flow group). Panel (H) shows gene expression of anti-apoptotic (BCL2) and pro-apoptotic (BAX) (normalized to GAPDH) at day 2 in SCBCs with qRT-PCR analysis is higher in flow groups than no flow groups (n=3, ** P<0.01 versus no flow group). Panel (I) shows hypoxia induced factor-1a (HIF-1a) expression at day 1 in the SCBCs in the ceMED (n=3, *P<0.05 versus no flow group).

FIG. 4 shows that subcutaneously transplanted EqC demonstrates equilibration with interstitial glucose resulting in increased cell viability in vivo. Panel (A) indicates that subcutaneously implanted EqC shows that high inflow glucose (20 mM) can be transported across the HF into the interstitial fluid (ISF). In determining optimal flow rates for in vivo studies, outflow glucose is found to be improved equilibrated with blood glucose at lower flow rates (250 μL/h) (n=3 rats, ** P<0.01, *** P<0.001 between blood and outflow glucose). Panel (B) shows EqC can capture the changes in interstitial glucose and equilibrate in a timely manner at 250 μL/h flow rate. When inflow glucose (2 mM) is lower than ISF glucose, glucose moves within a fluid into the EqC and outflow glucose increases demonstrating that the HF can capture ISF glucose. The outflow glucose reflects the expected peak delay after IP injection of glucose solution (2 g/kg) (n=3 rats). Panel (C) shows that for in vivo transplantation, a ceMED with dual EqC was designed: the first EqC detects changes in glucose in surrounding tissues and the second EqC allows insulin release into surrounding tissues. Panel (D) shows representative images and quantification of TUNEL positive cells showing that SCBCs housed in ceMEDs have higher viability than no flow at 14 days post-transplantation in vivo (n=4 rats, ** P<0.01 versus no flow group). Panel (E) shows retrieved SCBCs extracted from the core (near a HF) of a CC in the flow enhanced group expressed key endocrine markers (C-peptide, glucagon, and somatostatin) after extraction from the subcutaneous site of the rat at 7 days post-transplantation. Panel (F) shows SCBCs explanted from devices at 14 days post-transplantation show higher human insulin secretion in flow group (n=4 rats, *P<0.05 versus no flow group).

FIG. 5 shows that subcutaneously transplanted ceMED generates improved hyperglycemia reversal and lower fibrotic response in vivo. Panel (A) shows that blood glucose levels decreased at day 2 in the flow group in STZ-induced immunocompetent Lewis rat, and continued to 30 days post-transplantation. The ceMED is loaded with MIN6 cells (1.2×10⁷ cells/kg, n=3˜4 rats, *P<0.05, ** P<0.01, *** P<0.001 versus No device, ##P<0.01, ## #P<0.001 versus No flow). Panels (B) and (C) show that rats in the flow group showed less fluctuation and better restoration in glucose concentrations in response to intraperitoneal glucose tolerance test (IPGTT) compared to no flow and no device groups. The ceMED is loaded with MIN6 cells (n=3˜4 rats, ** P<0.01, *** P<0.001 versus No device, #P<0.05, ##P<0.01 versus No flow). Panel (D) shows primary human islets in flow group shows continuous reduction in non-fasting glucose level after transplantation. When the flow was stopped, an increase in blood glucose was observed (n=4 rats, *** P<0.001 versus No device, #P<0.05, ##P<0.01 versus No flow). Panel (E) shows representative images of retrieved primary human islets extracted from the core of a CC expressed C-peptide and glucagon by immunofluorescence staining. Panel (F) shows immunofluorescence staining which shows macrophage (CD68) and fibrosis markers (smooth muscle cell a (SMCa) and Collagen Type I (Coll)) on the surface of ceMED. Panel (G) shows representative images of histological staining (top: Hematoxylin and Eosin, bottom: Masson's trichrome staining) of device membrane extracted from the subcutaneous site of the rat show less fibrosis in the flow enhanced device. Panel (H) shows quantification of the fibrotic tissue thickness with flow compared to no flow devices show decreased thickness of fibrosis in the flow enhanced group (58.6% versus no flow group) [15 images total from 4 rats (4 devices) combined, *** P<0.001 versus No flow].

FIG. 6 shows dimensions of a 2D model of the device used for mathematical modeling.

FIG. 7 shows how selection of a hollow fiber with 100 kDa molecular weight cut-off allows for exchange of glucose and insulin while also protecting against passage of immunoglobins.

FIG. 8 shows how considering flow rates at 100 and 250 μL/h showed no significant differences with regards to glucose recovery percentage in the cell chamber (CC) after 10 and 15 min equilibrium in vitro (n=3). N.S., not significant.

FIG. 9 demonstrates that ceMED shows increased viability and glucose-stimulated insulin secretion (GSIS) indices of encapsulated stem cell derived ß clusters (SCBCs). Panel (A) shows the viability of cells in the core of ceMED (near HF) was evaluated using a LIVE DEAD kit after 3 days in culture. Panel (B) shows immunofluorescence images of SCBCs from the core of a CC expressed Nkx6.1 and C-peptide after 7 days in culture. Panel (C) shows cumulative GSIS indices from the SCBCs are significantly induced in the flow group than no flow group (n=3, *P<0.05 versus no flow group).

FIG. 10 shows SCBCs embedded in hydrogel with perfused flow. Panel (A) shows gross morphology of SCBCs in the CC with and without hydrogel. SCBCs generate large aggregates without hydrogel after 3 days in culture. Panel (B) shows gene expression profiles of anti-apoptotic (BCL2) and pro-apoptotic (BAX) (normalized to GAPDH) in SCBCs embedded in alginate gel and Matrigel (n=3).

FIG. 11 shows that a perfused flow through the equilibrium chamber (EqC) can increase SCBC viability. Panel (A) shows schematic images of SCBCs loaded in CC for 3 experimental groups. (a) the CC is perfused with and submerged in SCBC culture media (positive control). (b) the CC is perfused with and submerged in phosphate-buffered saline (PBS) (negative control). (c) the CC is submerged in PBS and connected to an EqC which is submerged in culture media in a separate chamber and perfused with PBS. Panel (B) shows a gross view of in vitro setup of experimental group (c). Panel (C) shows results of a LIVE DEAD assay which shows the importance of EqC for SCBC viability in the CC. Panel (D) shows gene expression ratio of BCL2 and BAX at day 3 in SCBCs loaded in CCs from (a)˜(c) [n=3, ** P<0.01 versus (b)].

FIG. 12 shows an animal swivel cage model including an external syringe pump to accurately control flow rate. Tether system allows for continuous infusion in live, active animals subcutaneously transplanted with ceMED.

FIG. 13 shows how a dual EqC can increase equilibration through the membrane and reduce outflow recovery. Panel (A) shows a pilot experiment setup comparing single EqC and dual EqC using 250 μL/h flow. The first EqC is submerged in artificial blood to visualize outflow or FITC-dextran (4 kDa) to quantify recovery. The second EqC submerged in PBS is incorporated in the dual EqC. Panel (B) shows visualization of outflow using artificial blood shows dramatic color loss in the dual EqC group. Panel (C) shows how the dual EqC reduces outflow FITC-dextran recovery compared to the single EqC (n=3, *** P<0.001 versus single EqC group).

FIG. 14 shows how subcutaneously implanted devices show presence for blood vessels on the surface after 14 days of implantation.

FIG. 15 shows that subcutaneously transplanted ceMED with human primary islets generates improved glucose tolerance in vivo. Panel (A) shows that rats showed faster glycemic control after intraperitoneal glucose tolerance test (IPGTT) (2 g/kg) when transplanted with ceMED (n=3˜4 rats). Panel (B) shows that a change in blood glucose from 30-60 min is higher in no device and no flow transplanted rats compared to rats transplanted with ceMED. Decrease in blood glucose levels from peak blood glucose to 30 min after (60˜90 min) IP injection, is greater in flow groups compared to no flow and no device group (n=3˜4 rats). Panel (C) shows that human insulin levels detected following the transplantation is higher in flow group compared to no flow (n=3 rats, ** P<0.01 versus No Flow group).

FIG. 16 shows that a polydimethylsiloxane (PDMS) based ceMED can accommodate the device for cryo-sectioning and surgical implantation.

FIG. 17 shows a closed-loop recirculation system using a peristaltic pump. Panel (A) shows a gross view of a closed-loop recirculation system. Panel (B) shows a schematic illustration of an experimental set up for measuring equilibration of CC in a closed-loop system. Panel (C) shows a perfused flow using peristaltic pump increased FITC-dextran (4 kDa) equilibration in the CC compared to no flow group (n=3, ** P<0.01 versus No flow).

FIG. 18 shows a diagram which depicts how a large surface area can be contained in a small implantable volume through the use of folding.

FIG. 19 shows bundling of tubular rosettes into a trocar-implantable geometry minimizes device Implant Area (the Origami Macroencapsulation Device).

FIG. 20 shows a CAD schematic of 4-petal rosette cross-section. Each “islet cubbie”-forming petal has an ID of 0.2-0.22 mm. The total ID of the rosette is 0.5-0.51 mm. Assuming 50 μm-thick membrane, the total OD of the rosette at its largest spans is ca. 0.6 mm.

FIG. 21 shows a graph of implant area vs total encapsulated islets in standard TC vs Origami Macroencapsulation Device (Folded TC).

FIG. 22 provides a diagram of a print-coating process.

FIG. 23 shows use of a high temperature 3D printer for printing molten sugar glass (sacrificial scaffolds).

FIG. 24 shows optimization of 3D-printed sugar glass. Panels (A and B) show substrate determination. Molten sugar glass removes cleanly from copper and aluminum substrates (Panels A, C, respectively), but adheres to silicon (Panel B). Panel (C) shows horizontal sugar glass printing. Panel (D) shows vertical extrusion (nozzle visible at top). Panel (E) shows a CAD schematic of 4-petal rosette. Rosettes may be achieved via horizontal printing or vertical extrusion. Panel (F) shows a custom-machined stainless steel nozzle for extrusion of 4-petal rosettes (top left) side view showing Luer lock, (top right) view of nozzle tip. (bottom) close-up of nozzle tip. 1 cm scaffolds may be printed or extruded within seconds.

FIG. 25 shows poly-E-caprolactone coating of extruded sacrificial scaffold. Panel (A) shows a sacrificial scaffold (sugar glass, formed by vertical extrusion). Panel (B) shows mounting of scaffold onto rotor (prior to coating). Panel (C) shows a poly-£-caprolactone-coated scaffold (formed by electrospinning). Scaffolds may be coated in bilaminar membranes within minutes.

FIG. 26 shows removal of sacrificial scaffold (sugar glass dissolution in ddH₂O). Panel (A) t=0 min, Panel (B) t=25 sec, Panel (C) t=1 min, Panel (D) t=2 min (complete removal).

FIG. 27 shows how coated membranes retain sacrificial scaffold architecture. Light microscopy of poly-£-caprolactone membrane formed over sugar glass scaffold with a spike feature (shown after sugar rod removal). The coated membrane retains the shape of the spike. Membrane thickness ca. 45 μm. Membranes were formed by electrospinning. Lumen diameter ca. 500 μm.

FIG. 28 shows how conformal poly-E-caprolactone membrane coating recapitulates TC membrane architecture. Membranes are directly deposited onto the surface of the sacrificial scaffold in two layers to recreate the bilaminar structure of the TC membrane. (Left) SEM of TC membranes. (Right) SEM of analogous, bilaminar PC membrane. (Top) The outer layer promotes vascularization with thin struts and large pore sizes. (Bottom) The inner layer affords alloprotection with small pore sizes. Membranes were formed by electrospinning.

FIG. 29 shows a completed single-rod device.

FIG. 30 shows a scaled-up system for mass production.

FIG. 31 shows a schematic of high-throughput hydrogel bundling factory with an example of an assembled trocar-implantable bundle of origami rosettes.

FIG. 32 shows SCB clusters loaded into prototype device units by gravity packing.

FIG. 33 shows that cells survive and maintain function during in vitro encapsulation challenge. Panel A shows a paraffin H&E longitudinal device section with healthy SCB morphology. Panels B, D, F show frozen H&E device sections which show healthy SCB morphology immediately following loading (Panel B), after 1 day of in vitro culture (Panel D) and after 5 days of in vitro nutrient deprivation (Panel F). Panels C, E, G show frozen insulin-DAB hematoxylin sections show encapsulated SCBs have healthy insulin-expression pattern within the cluster at the aforementioned timepoints.

FIG. 34 shows a schematic of a hydrogel scaffold device to support folded device units.

FIG. 35 shows a schematic of hydrogel-bundling mold.

FIG. 36 shows an example of a process for fabricating a cell encapsulating implantable device in accordance with some embodiments of the disclosed subject matter.

### DETAILED DESCRIPTION

In accordance with some embodiments of the disclosed subject matter, apparatus and methods for making such apparatus, for a cell-encapsulating implantable device are provided herein.

Current treatments for Type 1 diabetes (T1D) rely primarily on exogenous insulin replenishment via local injection. An alternative approach, namely whole pancreas or B-cell islet transplantation, either allogeneic or xenogeneic, has the potential advantage of sustained insulin production to restore normoglycemia, but cell sources are scarce and require life-long immunosuppressants, which exhibit long term side effects. To avoid immunosuppression, encapsulation approaches have focused on achieving immune-isolation of transplanted cells. Macroencapsulation is an encapsulation approach that has focused on achieving immune-isolation of transplanted cells by permitting a population of cells to act in synergy and to be contained in a single durable immune protective device at the desired site of implantation, thus facilitating retrieval. In this approach, insulin-secreting ß-cells can be encapsulated within a semipermeable membrane, creating a bioartificial pancreas that permits diffusion of oxygen, glucose nutrients, waste products, and insulin, but inhibits the penetration of immunocompetent cells or immunoglobulin which could immunologically damage the transplanted cells. To date, macroencapsulation devices have primarily relied on passive diffusion of oxygen and glucose which is restricted by the diffusion limit in tissues (˜150 μm).

Thus, an aggregate of cells enclosed within a device, without sufficient mass transport of oxygen and nutrients will quickly develop a necrotic core which has hindered this strategy from successfully translating to the clinic. Furthermore, the limited surface diffusion can also reduce the islets' sensitivity to fluctuations in patient's glucose level and thus delay the responsiveness in insulin secretion. There is a critical unmet need for a device that can accommodate a large number of islets and that can be successfully transplanted into a patient without significant cell death. For effective long-term treatment of T1D, it is extremely important to develop an immuno-isolation device with efficient cell packaging. State-of-the-art macroencapsulation devices (including certain commercial devices such as TheraCyte) suffer from geometric limitations that make them too large and prevent them from holding enough cells in surgically-feasible implants.

Specifically, device geometries of existing devices are limited to 2D wafers to ensure encapsulated cells are within the diffusion distance of the capsule surface because cells situated beyond this distance cannot receive nutrients from the host and die. For example, the implant area, or subcutaneous area occupied by a state-of-the-art macroencapsulation device capable of treating an adult Type I diabetic, is ca. 100-200 cm², which is too large for surgical implantation.

Embodiments of the disclosed device provide compact, 3D devices capable of housing enough cells to treat T1D patients in a surgically-feasible implant. One method is accomplished through an Origami Macroencapsulation Device (oMED): a Theracyte-like device “folded” into a condensed 3D shape, which leads to a device small enough to implant that supports sufficient islets to treat adult T1D patients. We have designed, mathematically modeled, and computationally optimized origami MEDs, arriving at a trocar-implantable design with dimensions small enough to implant in rodents (cylinder radius 0.7 cm, length 5.94 cm, implant area of 8.31 cm^{2 [91.7}% reduction from state-of-the-art device]). The design is modular and highly customizable to the patient and application desired. Another method to overcome cell survival loss, by minimizing the size of the device, is through the introduction of perfusate within the device. This method ultimately, enhances mass transport within the device for delivery of oxygen and glucose.

Convection enhanced MED: A convective transport system is integrated using a hollow fiber through the cell encapsulating device, the convection enhanced cell encapsulation device (ceMED) can accommodate increased cell density, viability and faster on and off insulin secretion compared to current macro-encapsulation devices. ceMED features two chambers, an equilibrium chamber (EqC) that interacts with surroundings and a cell chamber (CC) that houses immunoprotected cells through a polytetrafluoroethylene (PTFE) membrane.

Perfused flow primed with the conditions in the surrounding tissue equilibrates in the EqC and is then guided with a cylindrical hollow fiber into the core of an expanded islet layer for a continuous supply of nutrient exchange. A convection enhanced device allows for a higher density of islets to be loaded in the CC without compromising cell viability or transport of nutrients. The ceMED effectively captures the dynamics of glucose in surroundings to supplement the encapsulated islets with higher glucose sensitivity and faster insulin secretion on/off responses.

In various embodiments an origami MED is a human-sized MED small enough to be implanted and tested in rodents and is a MED that is small enough to be trocar-implantable, and includes an intricate 2D membrane “folding” to form compact and intricate 3D structures and optimized 3D geometries (including for example, 4-petal optimized modular rosettes).

Print-Coating Process: Print coating is a 2-step process for fabricating medical devices, particularly macroencapsulation devices.

We can 3D print MED capsules with complex geometries and features with procedures such as high temperature 3D printing (hence the term “print” in “print coating”). No prior MED has been 3D printed with sacrificial and/or non-sacrificial polymer inks/components which act as space-holders for encapsulated cells and/or materials and can be removed upon completion of the MED. Resulting vacant spaces in the capsule are subsequently filled with cells and/or materials. In the case of non-sacrificial components (i.e. PDMS) that are preserved in the finalized MED, these components can be used as matrices for cell cultures, structural supports, compartment dividers. perfusion channels, etc. Non-sacrificial components may also be embedded with chemical sensors, oxygen-eluting salts (CaOz) and reservoir growth factors (e.g., VEGF) or drugs (e.g., immunosuppressants), which may be 3D printed or added following fabrication. 3D printing allows for unprecedented precision, scalability, and ability to rapidly iterate design and features.

We can conformally coat 3D printed MED capsules with rotary jet sprayed membranes (hence the term “coating” in “print coating”) Membranes are coated directly on the surface of capsules, conforming to the shape of the capsules. Membrane properties (porosity, thickness, strut diameter+alignment, material composition) can be controlled with spray-coating parameters (polymer viscosity and molecular weight, injection velocity, rotor velocity, distance between nozzle and substrate, duration of spray, etc.), and are replicable. Multi-layer membranes (containing immunoprotective (nanoporous), vascularizing (macroporous), and supporting mesh layers) can be fabricated on the device surface without additional adhesives or deadspace. Ultimately, this process is scalable: appropriate spray coater could encapsulate 1000s of devices within seconds. Spray coater and 3D printer could be a single automated machine for industrial production.

We believe that we are well positioned following completion of this project to develop a device that will be ready for GMP pre-clinical assessment. While we have focused on using infusion pumps in our proof of concept work, as we move towards the clinic, we envisage using implantable refillable pumps that are already approved for human use as insulin pumps or for other applications. Improved mass transport may result from incorporating an internal recirculating pump such as a micro piezoelectric pump (Debiotech) or modified micro-peristaltic pump (similar to iPrecio). Owing to the vast clinical need, upon completion of this research, we hope that we are in a position where the developed device can be quickly translated to the clinic. We also envision potential combinations of both the OMED and ceMED incoporated into one device in which origami structures are present to conduct a perfusate through the device for convective nutrient delivery (active delivery).

In another embodiment, origami structures are produced by a print-coating method in which internal structures are patterned with a removable scaffold and immunoisolating components are patterned on the surface by methods such as but not limited to 3D printing, electrospinning, etc.

Islet transplantation for type 1 diabetes treatment has been limited by the need for lifelong immunosuppression regimens. This challenge has prompted the development of macroencapsulation devices (MEDs) to immunoprotect the transplanted islets. While promising, conventional MEDs are faced with insufficient transport of oxygen, glucose, and insulin due to reliance on passive diffusion. Hence, these devices are constrained to 2D wafer-like geometries with limited loading capacity to maintain cells within a distance of passive diffusion. We hypothesized that convective nutrient transport could extend the loading capacity while also promoting cell viability, rapid glucose equilibration, and physiological levels of insulin secretion. Here, we showed that convective transport improves nutrient delivery throughout the device and affords a 3D capsule geometry that encapsulates 9.7-fold more cells than conventional MEDs. Transplantation of a convection-enhanced MED containing insulin-secreting B-cells into immunocompetent hyperglycemic rats demonstrated a rapid, vascular-independent glucose-stimulated insulin response resulting in early amelioration of hyperglycemia, improved glucose tolerance, and reduced fibrosis.

Type 1 diabetes (T1D) is characterized by the autoimmune destruction of pancreatic B cells and burdens millions worldwide. T1D patients typically require life-long administration of insulin or immunosuppressive agent if received transplantation. Macroencapsulation device (MED) acts as a bioartificial pancreas and can immunoprotect encapsulated B cells. However, conventional MED suffers from limited cell loading capacity and slow glucose-stimulated insulin secretion (GSIS) due to sole reliance on diffusion. Here, we developed a convection-enhanced MED (ceMED) to afford 3D capsule geometry for maximized cell loading and faster GSIS driven by convection. Overall, we demonstrated that the ceMED significantly improves nutrient exchange that enhances cell viability and GSIS, ultimately leading to a rapid reduction of hyperglycemia.

Diabetes mellitus currently burdens over 387 million people worldwide, of which 5˜10% are accounted by patients with type 1 diabetes (T1D). T1D is characterized by the immune destruction of insulin-secreting B-cells and loss of glycemic regulation. Although intensive insulin injection regimens and the use of glucose monitors have been shown to effectively regulate blood glucose, patients are still unable to meet glycemic control targets. In particular, those with severe hypoglycemic events and glycemic lability cannot be effectively stabilized with these technologies. In 2000, the Edmonton protocol was developed as a procedure that directly infuses pancreatic islets, isolated from cadaveric donors, into the portal vein to treat unstable T1D. This procedure led to insulin-independence in patients for a short period post-infusion. However, poor long-term graft survival due to alloimmune and autoimmune rejections and engraftment inefficiency prevents sustained therapeutic effects. Although immunosuppressants are co-administered with the transplanted cells to prevent graft rejection, 56% of patients experience partial to complete graft loss after 1 year, and only 10% of patients remain insulin-independent after 5 years. The majority of patients also experience complications from immunosuppression, including elevated risk of opportunistic infections and cancer. In addition, islet transplantation is burdened by a major islet donor shortage since often two or more human pancreases are needed to achieve a sufficient number of islets.

The problems of immune rejection could be overcome with macroencapsulation devices (MEDs), in which glucose-sensing-insulin-secreting cell sources like pluripotent stem cell-derived ß clusters (SCBCs) or other islet sources, are implanted within an immune-isolating vehicle to promote cell survival and function. In MEDs, islets are housed in a single compartment that selectively permits the exchange of nutrients while obstructing host immune effectors such as cells and antibodies. Over the past few decades, MEDs have successfully restored insulin independence and normoglycemia in T1D animal models. However, scaling these devices for human applications has been challenging. Currently, passive diffusion based MEDs including Encaptra, βAir Bio-Artificial Pancreas, Cell Pouch™, and MAILPAN® are being explored in phase I/II clinical trials. Nevertheless, these diffusion-based devices still suffer from limitations in transport of glucose, insulin and other biomolecules to the core of these devices, which compromise the survival and function of encapsulated cells. Ultimately, these devices are restricted in geometry, thickness, and cell loading capacity.

More specifically, a significant portion of encapsulated cells become non-viable immediately after transplantation due to lack of vascularization, which results in hypoxia and limited nutrient availability. Thus, during the initial pre-vascularization period, which lasts approximately 14 days post-transplantation, solute exchange and insulin secretion cannot occur effectively using conventional MEDs. For this reason, many encapsulated cells prematurely lose their function and eventually die. Various strategies have been developed to expediate angiogenesis around the device, especially during the initial hypoxic period after device implantation, to reduce cell loss. Examples include early vascularization of device, infusion of vascular endothelial growth factor, and co-transplantation of mesenchymal stem cells (MSCs). In another instance, βAir Bio-Artificial Pancreas incorporated a daily-refillable oxygen chamber in between two islet slabs to maintain adequate oxygen supply, but the chamber is 15 to 30-fold thicker than islet layers. Despite improvements in cell viability, this strategy still cannot guarantee adequate glucose sensing and insulin release kinetics of the islets, and further limit the available space for cell packing.

To supply cells with enough nutrients, it has been suggested that islet density of the MEDs should be set to 5˜10% of the volume fraction. Consequently, a limited mass of islets must be placed within a large device to ensure optimal nutrient distribution. Otherwise, devices exhibit extreme cell loss. For instance, TheraCyte™, which packs 70˜216 islet equivalent (IEQ) in 4.5 μL or 1,000 IEQ in 40 μL volume, exhibited poor cell survival. The remaining cells were neither capable of restoring euglycemia in rodents (1,000˜2,000 IEQ required) nor sustaining a therapeutic dosage needed for humans (˜500,000 islets) in a reasonably sized device.

To overcome nutrient delivery challenges and improve cell loading capacity, we designed a convection-enhanced macroencapsulation device (ceMED) to perfuse the device continuously, thus providing convective nutrient transport. We hypothesized that a ceMED with a continuous flow would: i) transport more nutrients compared to passive diffusion-based devices, ii) increase the cell density and survival beyond the distance limit for diffusion, iii) support a three-dimensional (3D)-expanded cell layer to increase the loading capacity, iv) improve glucose sensitivity and timely insulin secretion via faster biomolecule transport in and out of the device, and v) show efficacy in vivo by reducing hyperglycemia before vascularization. Overall, we demonstrated that the convective motion promotes survival of insulin-secreting B cells encapsulated at high density. We also demonstrated that it effectively captures the dynamics of glucose concentrations in the transplantation site, resulting in more appropriate insulin secretion with faster on/off responses. Finally, the ceMED showed early reduction in blood glucose levels in hyperglycemic rat models several days prior to the critical 14 days post-transplantation.

Convection Enhanced Macroencapsulation Device (ceMED) Design

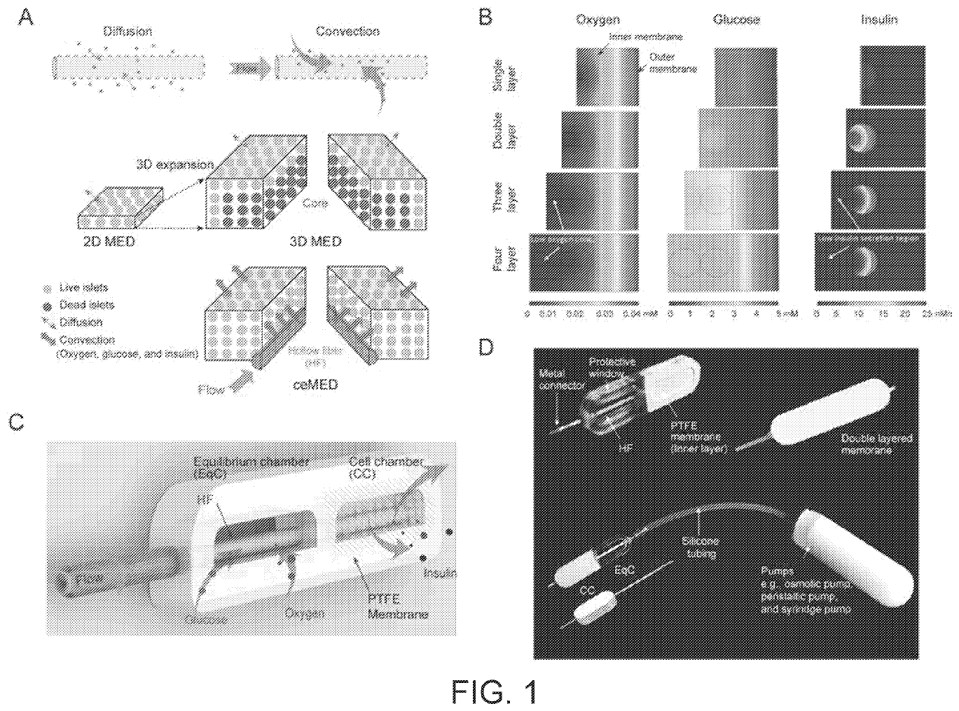

Convection-based nutrient exchange system can potentially solve many of the problems faced when using diffusion-based system. Convection can provide a more active and faster transport of fluid, both solute and solvent, throughout the device (FIG. 1A). On the other hand, diffusion only provides a steady transfer of solutes along a concentration gradient at the surface lining the device. Hence, diffusion-dependent macroencapsulation devices (MEDs) inevitably experience cell death due to limited nutrient exchange and increased hypoxia within the inner most layer of cells (FIG. 1A). To determine the limitations of diffusion-based nutrient transport in MEDs, we computationally simulated nutrient transport within a conventional device (TheraCyte™) (FIG. 1B, see below). Mass transport and flux of oxygen, glucose, and insulin through the capsule membranes were calculated for devices of varying thickness (FIG. 1B). While a monolayer of islets received a sufficient amount of nutrients for survival, the presence of multiple layers of cells resulted in insufficient oxygen availability (oxygen concentration <0.0001 mM) to support insulin secretion and cell survival of the inner layers. Only the outer layer of cells was predicted to function and secrete insulin in such a multilayer device. These simulation results demonstrate the need for active nutrient transport systems to enable high density packaging of cells in a 3D configuration.

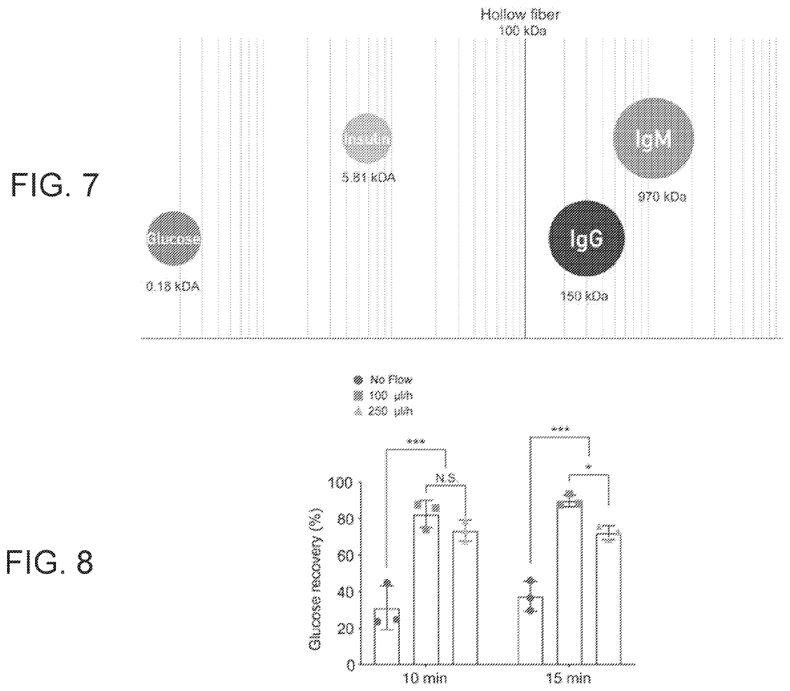

Thus, we designed a ceMED to provide convective nutrients through a continuous flow of fluid to the encapsulated cells (FIG. 1C). In some embodiments the ceMED features two chambers, an equilibrium chamber (EqC) that collects nutrients from surroundings and a cell chamber (CC) that houses immunoprotected cells enclosed within bilaminar polytetrafluoroethylene (PTFE) membranes (200 nm) (FIGS. 1C and 1D). Based on immune protection strategies used by TheraCyte™, the outer membrane promotes angiogenesis while the inner membrane selectively allows nutrient transport and protects against immune onslaught. The perfusate is guided via a cylindrical semipermeable hollow fiber (HF) from the EqC, where it is primed with the condition in the surrounding tissue, into the core of an expanded layer of insulin-secreting B cells, providing a continuous supply of nutrients. Specifically, a molecular weight cut-off (MWCO) of 100 kDa was used such that it selectively passes essential nutrients, including glucose and insulin (<100 kDa), while protecting islets from small molecular effectors of the host's immune system (150 kDa˜900 kDa) (See below and in FIG. 7). By the time the perfusate has reached the CC inlet, it will be primed with a similar concentration of nutrient as the subcutaneous interstitial tissue surrounding the implant.

Investigation of optimal loading conditions and parameters of a ceMED in vitro

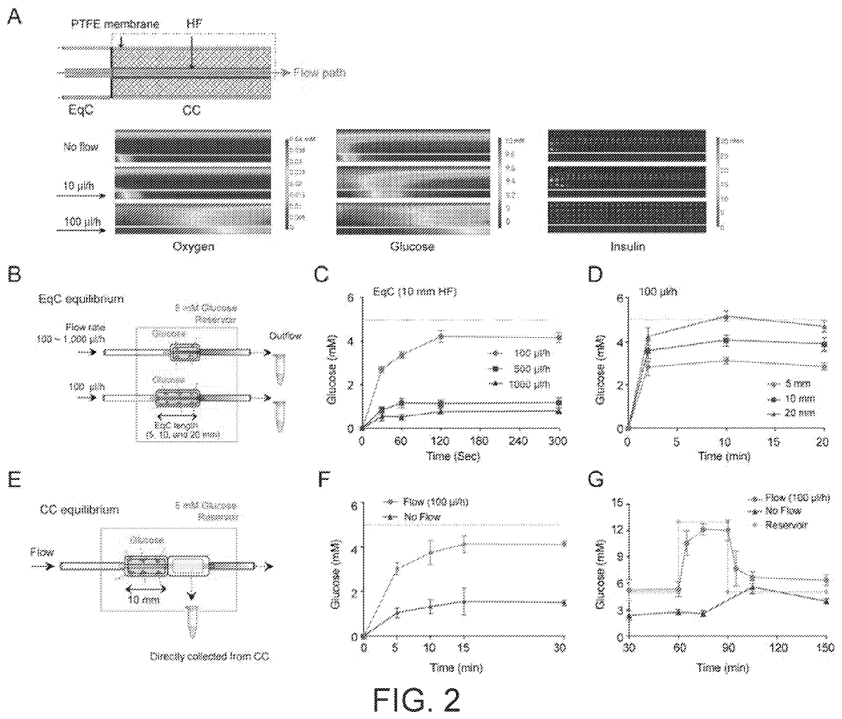

Optimal device parameters were estimated with computational models of convective transport in the prototype ceMED, which demonstrated that both glucose and oxygen transport increase as a function of flow rate. The convective transport also allowed nutrients to permeate the modelled device interior to supply islets situated beyond the diffusion limit from the membrane surfaces (FIG. 2A, see below). The model also predicts improved insulin secretion from encapsulated cells throughout the device proportional to increased glucose transport. Specifically, the simulation data predicts that, under static conditions (0 μl/hr flow rate), all islets of the inner layers are hypoxic. Whereas even a low flow rate (10 μl/hr) modestly increases oxygen concentration for cells at the inlet, resulting in some insulin secretion. Flow rates greater than 100 μl/hr are predicted to sufficiently deliver oxygen and glucose to most encapsulated cells and afford uniform insulin secretion throughout the device. In order to maintain the same device conditions in both no flow and flow groups, HF is also introduced into the no flow device.

To test these predicted conditions in vitro, the prototype EqC was isolated and submerged in a reservoir of 5 mM glucose, which is comparable to the physiological basal blood glucose level. In order to validate the effectiveness of a flow-based convection at controlled flow rate, a syringe pump was used. The concentration of glucose present in the outflow was measured as a function of flow rate while the EqC length was held constant at 10 mm (FIG. 2B). As expected, the time to reach the plateau glucose concentration at the outlet was faster at higher flow rates. 100, 500, and 1,000 μL/hr groups achieved plateau at 120, 60, and 30 seconds respectively. However, this occurred at the expense of reduced glucose equilibration due to insufficient residence time of the perfusate within the HF. Glucose equilibration at 100, 500, and 1,000 μL/hr reached 84.2%, 23.6%, and 16.0% at the respective flow rates (FIG. 2C). To determine the relationship between EqC length (also denotes HF length) and glucose equilibration, we tested multiple EqC lengths (5, 10, and 20 mm) with a fixed flow rate of 100 μl/hr. As expected, longer HFs resulted in improved equilibration, likely due to increased surface area and time for solute transfer between the fluid in the HF and the reservoir (FIG. 2D).

To compare the glucose concentration delivered to encapsulated cells within the CC under diffusion versus convection-enhanced conditions, the complete prototype ceMED (EqC and CC without encapsulated cells) was submerged in the same test reservoir, and fluid from the CC was collected to determine its glucose concentration (FIG. 2E). At plateau for the experimental group with convection (˜15 min), the diffusion-based device (No flow) had 31.1% glucose equilibration, whereas perfusion at 100 μl/hr resulted in 82.5% glucose equilibration (FIG. 2F). Perfusion at 250 μl/hr flow resulted in 73.4% glucose equilibration and did not show a statistically significant difference compared to the 100 μl/hr flow rate after 10 min (See below, FIG. 8). However, significant decrease in glucose equilibration at 250 μl/hr compared to 100 μl/hr flow rate was observed after 15 min. This slight difference in equilibration is presumably due to the reduced residence time for nutrient exchange in the 250 μl/hr perfusion group. Altogether, our cumulative in vitro data suggest that the optimal flow rate for ceMED functionality lies within the range of 100˜250 μl/hr.

We additionally characterized the behavior of the CC in a simulated glucose tolerance test, in which the concentration of reservoir glucose was increased from basal state to post-prandial blood glucose concentration (5 mM to 13 mM to 5 mM, sequentially). The post-prandial state corresponds to the glucose concentration 30˜60 minutes after meal feeding. Following 5 to 13 mM reservoir transition, the ceMED glucose recovery in the 100 μl/hr flow group increased rapidly during the first 5 min (at 65 min mark, 10.6+1.4 mM) and then plateaued to 93% recovery by 15 min (at 75 min mark, 12.2+0.6 mM). Whereas the non-perfused device recovered only 43% of reservoir glucose in 45 minutes (at 105 min mark) following 13 mM reservoir. Following 13 to 5 mM reservoir transition, we also observed a rapid decrease in CC glucose concentration recovered in the first 5 min (at 95 min mark, 7.7+1.4 mM) and then plateau by 15 min (at 105 min mark, 6.7+0.7 mM) (FIG. 2G). The observed rapid on/off response in CC glucose in the first 5 min and steadying by 15 min following a change in reservoir glucose is consistent with the data shown in FIG. 2F. These results suggest the ceMED will better recapitulate the dynamic response of the endocrine pancreas by providing a more accurate concentration of interstitial solutes to encapsulated tissue in a more timely fashion.

ceMED Shows Increased Cell Viability and Higher Insulin Secretion Activity In Vitro

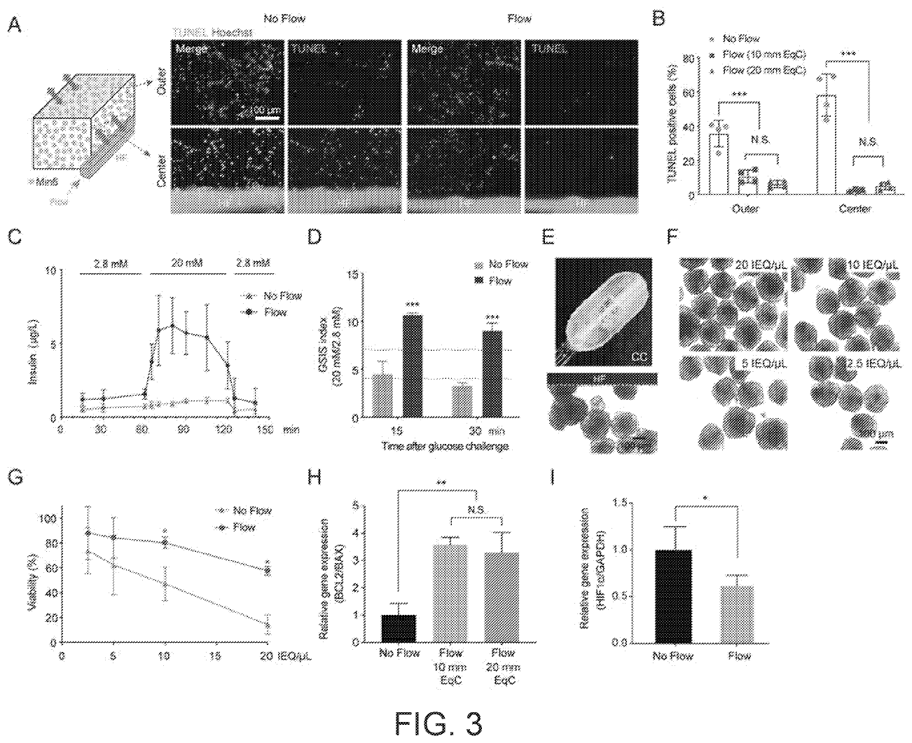

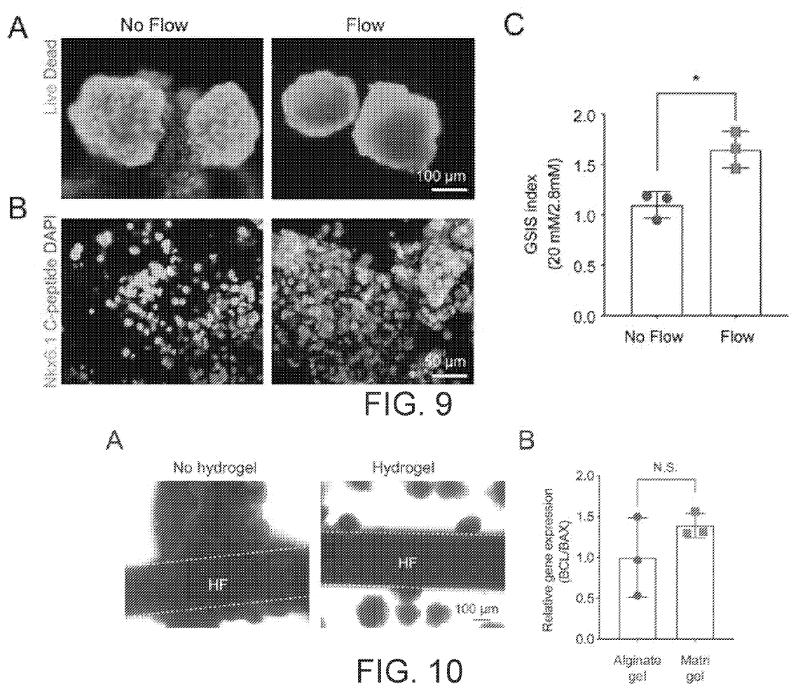

To investigate the viability and functionality of encapsulated cells in vitro, ceMEDs were first loaded with MIN6 cells. MIN6 cells were chosen due to their widespread use and physiological similarity to primary human B cells, as well as, for their robust glucose sensitivity and glucose-stimulated insulin secretion (GSIS) response. We hypothesized that convection and consequent nutrient delivery through the core of the device would result in improved viability, at both the outer and inner layer of cells. As expected, the reduction of TUNEL positive cells, especially surrounding the HF in the flow condition generated uniform cell viability throughout, whereas the no flow condition yielded more cell death (FIG. 3A). The number of apoptotic MIN6 cells at the surface and center of the CC was significantly increased in the no flow condition compared to the flow condition (FIG. 3B). No significant differences in viability were observed between 10 mm and 20 mm EqC lengths in either condition. To see whether flow could increase the on and off rate of insulin secretion, we checked the sequential levels of insulin secretion after immersion into glucose solutions at multiple time points (FIG. 3C). After 10 minutes of 20 mM glucose challenge, the change in insulin secretion by cells in the flow group increased by ˜2.4 fold, representing a rapid “on” insulin secreting response to glucose stimulation, compared to the no flow group, which did not increase. Similarly, flow-enhanced encapsulated cells exhibited a substantial decrease in insulin production (“off” response) by 3.6 fold, following a drop in glucose levels back to 2.8 mM. Whereas the no flow device exhibited a more gradual decrease in insulin production by 2.5-fold. In addition, the cumulative GSIS index (i.e., values at 15 and 30 minutes post 20 mM glucose challenge divided by 15 and 30 minutes post 2.8 mM glucose challenge, respectively) for the flow-enhanced group showed a statistically significant, 2.5-fold increase which is shown to be above the GSIS index range of static MIN6 cells (dashed lines) (FIG. 3D). Additionally, to further validate the cell viability and function of ceMED, LIVE DEAD and GSIS analyses were then conducted on the SC B cell clusters (SCBCs) loaded in the ceMED. Similar to the MIN6 cell-loaded ceMEDs, the perfused flow also induced cell viability and increased insulin secretion indices when loaded with SCBCs (See below, FIGS. 9A and 9C). Moreover, immunofluorescence imaging of the SCBCs cultured for 7 days in the CC revealed that the cells in the flow group retained spherical morphology and expressed Nkx6.1 and C-peptide markers. However, the cells in the no flow group did not retain the morphology and were more scattered (See below, FIG. 9B). Altogether, perfusion of the ceMED supports overall increased viability throughout the capsule thickness, and the 3D geometry allows highly increased cell capacity compared to no flow conditions. Moreover, convection also enhanced glucose sensing and insulin release kinetics, as well as total insulin delivered from the device after glucose stimulation.

ceMED Supports Viability of SC ß Clusters at an Increased Loading Capacity

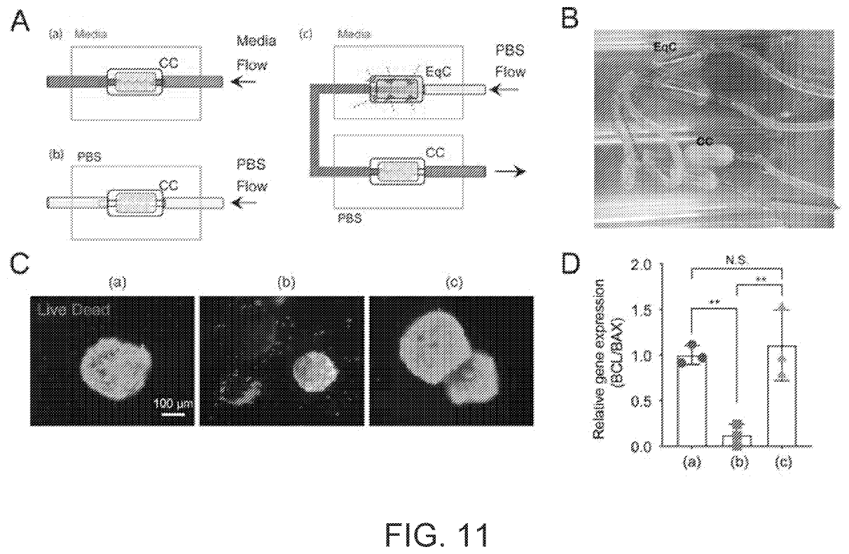

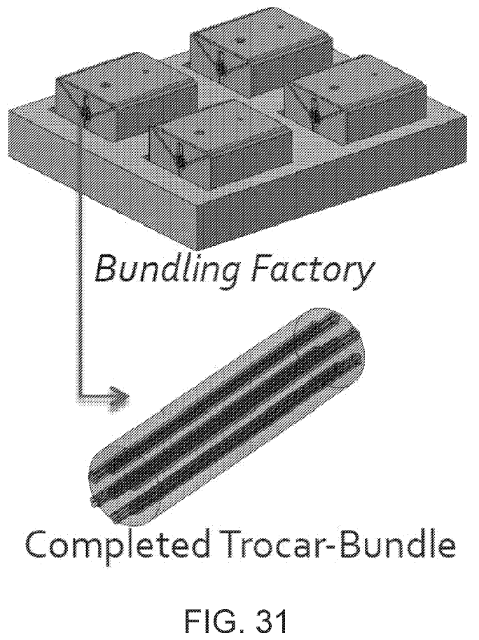

Next, we sought to explore the loading capacity of the ceMED with SCBCs, a more clinically relevant cell source. In the SC B protocol, SC B cells grow in 3D clusters, of which ˜30% are B cells that have the ability to secrete insulin in response to glucose. The cell morphology of SCBCs was not visibly affected after loading into the CC (FIG. 3E). Moreover, SCBCs were suspended in growth factor reduced Matrigel and injected into the CC. Matrigel was used for homogenous cell distribution, prevention of aggregates, and shear stress reduction during loading (See below, FIG. 10A). We also investigated cell loading into the ceMED with alginate hydrogel, wherein alginate microspheres are commonly used for islet encapsulation. The ratios of expression of the anti-apoptotic marker BCL2 and the pro-apoptotic marker BAX (BCL/BAX) showed no significant difference between the Matrigel-embedded and the alginate-embedded cells (See below, FIG. 10B). To determine the optimal packing density for viability and function, SCBCs were loaded into the CC at multiple densities of islet equivalents (IEQ: 20, 10, 5, and 2.5 IEQ/μL). Their viability and function were compared to the encapsulated cells without perfusion (FIG. 3F). The percentage of viable cells was determined by alamarBlue assay and normalized to a control population of SCBCs in suspension culture. After 2 days in culture, the flow-enhanced group loaded with 10˜20 IEQ/μL and perfused with PBS at 100 μl/hr maintained significantly higher numbers of viable SCBCs compared to the no flow condition (10 IEQ/μL: flow 81.0+4.5%, no flow 47.0+13.6%; 20 IEQ/μL: flow 57.5+3.6%, no flow 7.8+7.8%) (FIG. 3G). Considering 10 IEQ/μL as the optimal loading capacity, the ceMED can accommodate a 9.7 fold higher cell capacity with 81.0% viability compared to TheraCyte™ [ceMED: 1,621 IEQ/cm², dimension of dual membrane surface) versus TheraCyte™ (40 μl): 167 IEQ/cm²]. The BCL/BAX ratios further suggest enhanced viability for the flow conditions compared to no flow condition (FIG. 3H). However, no significant difference in viability was observed between 10 mm versus 20 mm long EqC. To assess contribution of EqC on the cell viability, we detached EqC and CC. SCBC-containing CC submerged in PBS was supplied with culture media via the tubing from the EqC, which is primed with the culture media (See below, FIGS. 11A and 11B). When an EqC was introduced into the circuit, it provided flow-enhanced equilibration with the surrounding media and conduced the primed PBS to the cells in CC. Subsequently, the addition of EqC resulted in a higher number of live cells and a BCL/BAX ratio comparable to the positive control (direct perfusion of culture media into the CC), suggesting the importance of the EqC in improving SCBC viability (See below, FIGS. 11C and 11D). Furthermore, we found that the level of hypoxia induced factor-1a (HIF-1a) expression in the flow-enhanced group was lower in the SCBCs than those in the no flow group (FIG. 31). The HIF-1a expression is induced by low oxygen concentrations and can modulate diverse signaling pathways involved in ß cell apoptosis.

Glucose Equilibration Across HF in Subcutaneously Transplanted ceMED and Increased Cell Viability In Vivo

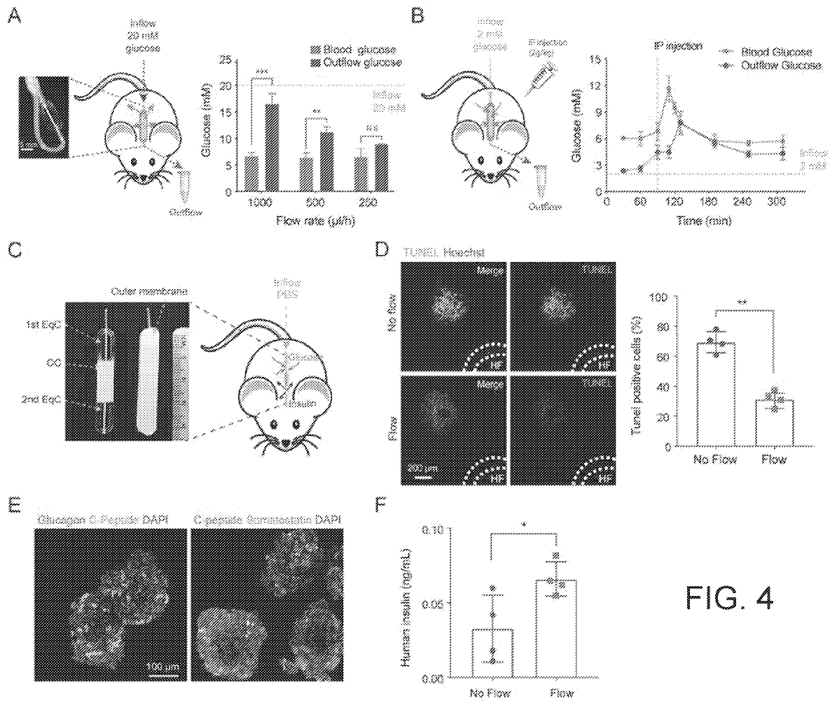

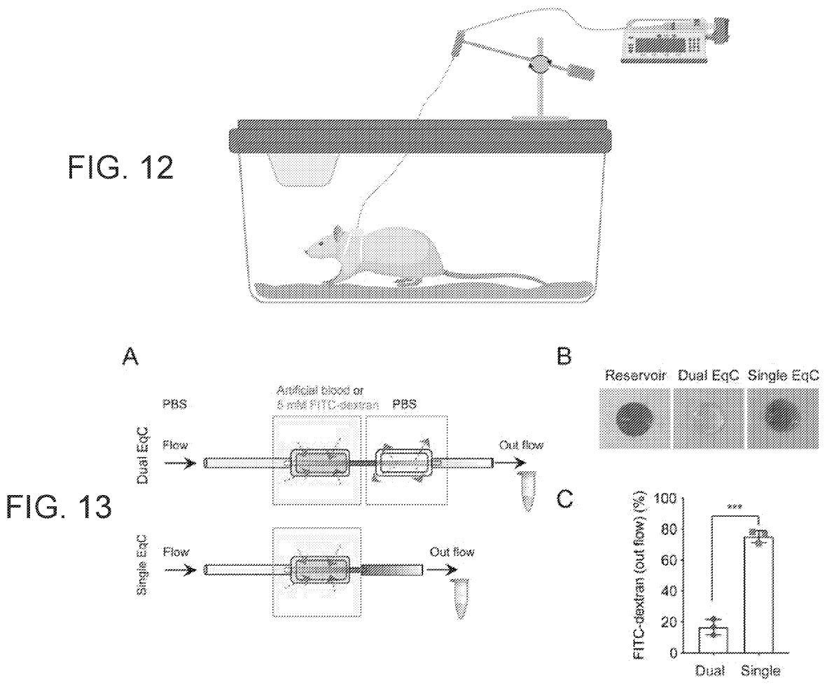

To examine the transport of glucose across the HF and equilibration under in vivo conditions, outflow glucose and blood glucose were measured while the EqC was implanted in the subcutaneous space of non-diabetic Lewis rats using swivel-based tether in vivo infusion set up (See below, FIG. 12). After surgical wound healing, to investigate whether bilateral transport could occur across the HF, high (20 mM) and low (2 mM) glucose concentrations were perfused through the HF. Inflow concentrations of 20 mM and 2 mM in PBS were chosen to clearly visualize bilateral transport using the largest possible range between satiated and starving glucose dynamics. Here, PBS was used as a perfusate because it has number of uses clinically and to maintain a constant pH. The initial target flow rate of 100 μl/hr proved ineffective at transporting the fluid through the significantly longer length of tubing required. Thus, we sought to re-confirm the optimal flow rate, starting at 250 μl/hr in vivo (250, 500, and 1,000 μl/hr) (FIG. 4A). When 20 mM glucose was infused, outflow glucose approached a concentration near normal blood glucose (5˜6 mM) with decreasing flow rate suggesting that inflow glucose equilibrated with ISF glucose. Among the groups, 250 μL/hr flow rate showed the best equilibration, which is consistent with our earlier in vitro data (FIG. 2C, see below, FIG. 8). From here on, 250 μl/hr flow rate was used for the in vivo assessments.

When 2 mM glucose was infused, outflow initially rose to equilibrate ISF concentrations (FIG. 4B). After 90 minutes, in which glucose completely equilibrated between the EqC and ISF, we artificially increased the glucose level in the rats by performing intraperitoneal (IP) injection of glucose (2 g/kg). IP injections in the rat immediately raised its blood glucose level to up to 12 mM and returned to normal within 40 min, with peak concentration around 30 minutes post IP injection (red line). The outflow glucose (blue line) manifested a similar tendency as ISF glucose dynamics in response to blood glucose fluctuations. Together, the two experiments revealed that transport of solutes occurs bidirectionally across the HF in vivo in the presence of a gradient between ISF and perfused solution.

In order to provide both an inflow glucose and outflow insulin equilibration region to deliver glucose to the encapsulated cells and insulin to the animal, the ceMED was modified with an additional EqC (FIG. 4C). A pilot study was performed to evaluate feasibility of using this modified device in vivo by comparing the performance between single EqC versus dual EqC. The dual EqC demonstrated profound solute release from the second EqC while reducing the solutes wasted through the outflow (See below, FIG. 13).

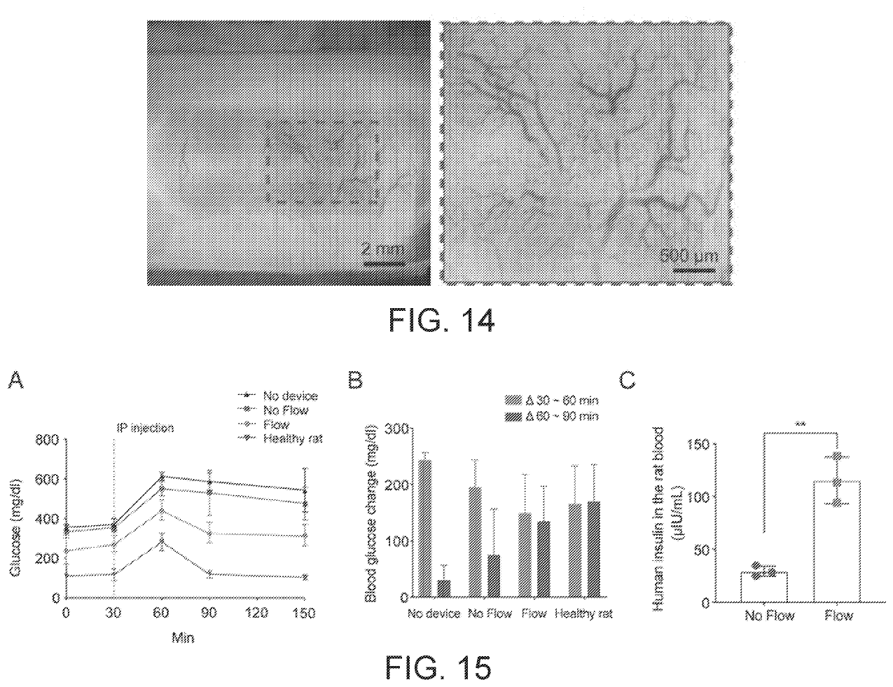

Following 14 days post-transplantation, the surface of the devices showed a high density of blood vessels surrounding the outer PTFE membrane (See below, FIG. 14), which is consistent with results from others. The flow-enhanced devices sustained a higher viability of SCβCs than non-flow infused devices as demonstrated by decreased TUNEL positive cells (Flow: 31.4+5.2% and No flow: 70.0+7.0%) (FIG. 4D). Then, the SCβCs retrieved from the implantation site were shown to express key endocrine markers including C-peptide, glucagon, and somatostatin (FIG. 4E). Proper functioning was shown by detecting human insulin levels at significantly higher levels than the no flow transplanted rats (FIG. 4F). Overall, it was validated that the device could effectively interact with surrounding ISF and equilibrate dynamic glucose changes. Furthermore, the device could establish extensive angiogenesis and maintain cell viability, particularly in layers near the HF.

Transplantation of Glucose-Sensing-Insulin-Secreting Cells within the ceMED to Restore Glycemic Control

We then investigated the short-term efficacy of the ceMED in supporting the viability and function of immune-isolated glucose-sensing-insulin-secreting cells, particularly during the pre-vascular 14-day period, in a chemically-induced hyperglycemic animal model. First, MIN6 cells (1.2×10⁷ cells/kg, assuming 1,500 cells=1 IEQ) embedded in an alginate hydrogel were loaded into the device at a seeding density similar to that of previously reported macroencapsulation studies, 6,500˜8,600 IEQ/kg. Then the device was transplanted into the subcutaneous space of Streptozotocin (STZ)-induced immunocompetent Lewis rats. Throughout the study, the control group did not receive infusion while the flow group underwent perfusion with PBS at the flow rate of 250 μl/h. In the flow group, blood glucose began to decrease as early as 2 days post-transplantation and reached near normoglycemia by day 5. This result demonstrates that the flow system may promote early cell survival and insulin secretion from the ceMED even before vascularization takes place (14 days post-transplantation). The flow group showed continued reduction of hyperglycemia with a mean non-fasting blood glucose of 187.0±32.9 mg/dl compared to no flow group (453.8±57.6 mg/dl) 25 days post-transplantation (Flow: 198.2±21.3 mg/dl and No flow: 495.5±44.8 mg/dl 30 days post transplantation, FIG. 5A).

After transplantation, intraperitoneal glucose tolerance test (IPGTT) was performed on day 14, following overnight fasting, in which the ceMED group showed enhanced glucose tolerance approaching near normal glycemic level (FIGS. 5B, 5C). Between the time of IP injection (30 minutes) and the time of peak blood glucose concentration (60 minutes), the untreated control group and transplanted rats with no flow group exhibited a steeper rise in blood glucose concentration (234±32.9 mg/dl and 208±38.4 mg/dl) compared to ceMED (193.7±11.9 mg/dl) transplanted rats. From the time of peak blood glucose (60 to 90 minutes), the flow group showed improved clearance of IP injected glucose with a greater decrease in blood glucose (A=−128±21.3 mg/dl), compared to no flow and no device groups (A=−62.0±46.0 mg/dl and A=−23.0±15.7 mg/dl) (FIG. 5C). These results also suggest that the ceMED generates less pronounced fluctuations in blood glucose concentrations.

To check human insulin secretion, primary human islets (8,000 IEQ/kg) were loaded into the device. Similar to the MIN6 cell study, the flow group showed more effective reduction of hyperglycemia by 2 days post-transplantation. To clarify the effect of perfused flow, the syringe pump was stopped 18 days after transplantation. Interestingly, when flow was stopped in ceMED transplanted rats, blood glucose increased from 277+44.1 (Day 17) to 449.0+134.2 (Day 24) (FIG. 5D).

IPGTT was also conducted on rats transplanted with primary human islets loaded in ceMED. Like MIN6 cells, primary human islet loaded in ceMED also demonstrated reduced blood glucose fluctuation after IP injection (See below, FIGS. 15A, 15B). Importantly, a 3.9-fold increase in human insulin in the flow group indicates that glucose reduction is regulated by insulin specifically produced by the transplanted primary human islets (See below, FIG. 15C). Furthermore, immunofluorescence imaging of the retrieved primary human islets revealed that cells in the flow group retained spherical morphology and expressed C-peptide and glucagon markers. On the other hand, the cells retrieved from the no flow group did not appear to retain this morphology (FIG. 5E). These data suggest that the perfused flow is essential in releasing insulin from the insulin secreting cells (C-peptide+) in the islets within the ceMED for maintaining their morphology and reducing blood glucose levels.

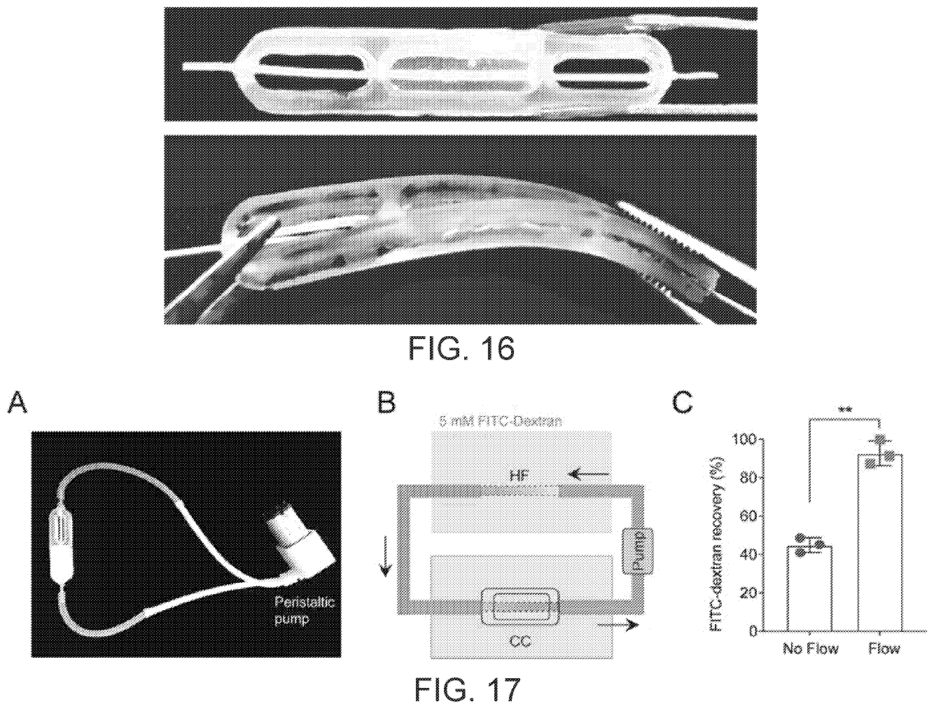

An interesting observation made from the in vivo study is that infused devices (flow group) elicited a lower fibrotic response on the retrieved PTFE membrane when compared to static group (no flow). This was demonstrated by the decreased macrophage (CD68) and fibrotic markers such as smooth muscle cell a (SMCa) and collagen type I (Coll)) (FIG. 5F). In order to section devices for histological staining, we developed a polydimethylsiloxane (PDMS) based device (See below, FIG. 16). The PDMS based device can not only accommodate sectioning, but also presents as a soft device for surgical implantation. Histological staining of fibrotic tissue adjacent to the CC membrane also showed decreased thickness of fibrotic tissue from ˜548 μm in no flow group to ˜321 μm in the flow group (FIGS. 5G, 5H).

Thus, as disclosed herein the glucose-sensing-insulin-secreting cells-loaded ceMED, as with many other cell therapies, provides unique advantages over conventional insulin pumps in that it acts as a biological glucose sensor that intrinsically monitors glycemic levels, produces insulin indefinitely, and secretes it on demand as needed. While we primarily demonstrated a proof of concept of the ceMED, we also demonstrated successful encapsulation and in vivo survival of multiple types of glucose-sensing-insulin-secreting cell sources. Moreover, we have shown that perfusion of the ceMED through a HF can compensate for the delay in vascularization after transplantation. This allows the device to sustain cell viability and decrease blood glucose level as early as 2 days post-transplantation.

An additional benefit of external perfusion is that it avoids complications such as blood clotting and thrombosis that may arise with intravascular-encapsulation devices. As opposed to most static diffusion devices, the ceMED can be transplanted into the less vascularized subcutaneous site which requires less invasive implantation surgery. Static devices are usually limited to transplantation sites with dense vascularization such as the peritoneal cavity or omentum. These sites are characteristically small, invasive to accommodate large-sized capsules and highly dependent on hemocompatibility. Static devices also have a suggested maximum loading density of 5˜10% of the total device volume in order to ensure adequate nutrient distribution. Whereas the ceMED can load cells to 23.5% of the total volume [13.3 IEQ/μL (maximum: 56.6 IEQ/μL, assuming 150 μm diametric islets)], while sustaining islet insulin secretion and viability in vivo. The increased cell loading capacity, under convection, suggests that the ceMED can maintain a smaller device size to achieve the same therapeutic effect resulting in a less invasive transplantation.

During the in vivo glucose equilibration test, our subcutaneously transplanted device shows similar dampened and delayed peak response to ISF dynamics following IP injection. We postulate that ISF glucose exhibited diminished and delayed equilibration with blood glucose since ISF glucose dynamics are known to be more stable than blood glucose and exhibit ˜30 minute delays and lower peak values compared to blood glucose levels. Alternatively, the long tubing associated with the setup of our infusion system could have contributed to the delay in outflow glucose concentration and the dilution effects within the tubing could confer the dampened peak.

While in vivo experiments with ceMED show enhanced capability in reducing elevated glucose levels, further work will be needed to extend the normoglycemic period for long-term. This may be achieved by further scaling up the devices to support greater numbers of cells. Specifically, future scaling up of cell loading capacity for clinical applications (500,000 islets) requires efforts in not only providing convection-based transfer, but also by forming a loading space with maximized surface area to volume ratios. Exploring structural modifications such as folding or coiling may potentially provide a solution for high-surface area and compact geometries tailored for clinical application. As a prototype, for the purpose of ceMED validation in a small animal model, the EqC structure was designed similar to the CC. Next generation ceMED may be designed so that the EqC will have no guide frames. Minimizing the volume occupied in the EqC may reduce the overall volume of the ceMED and further increase cell loading capacity.

There is another important reason why long-term in vivo assessment is necessary. The transplanted ceMED was able to overcome the critical pre-vascular period, as the device was able to produce insulin and reduce blood glucose before vascularization. However, the blood glucose levels in the same hyperglycemic animals increased again after the flow was stopped at day 18. Therefore, our short-term in vivo transplantation study showed that the flow-based convention system is crucial for the survival and functionality of islets in the pre-vascular phase and potentially in the long-term. In its first-generation embodiment, our device implements a one-way fluid flow system where the infusion pump pushes the fluid through EqC and CC sequentially. To eliminate the need for an external pump or the combination of an internal pump with a reservoir that needs periodic replenishment, a closed-loop recirculation system is important for next generation development. In addition, the long tubing and animal movements made long-term experiments difficult when using the syringe pump-mediated flow system. Because of this, ceMED functionality assessment in vivo was stopped at 30 days post-transplantation. Hence, examination of long-term ceMED functionality using an alternative pump with closed-loop system will be the focus of future work (See below, FIG. 17). Moreover, SCβCs represent useful cell source for in vivo ceMED validations, especially considering their clinical potential. However, limitations exist with validating ceMED using SCβCs. SCβCs are unable to secrete insulin until 2 weeks post-transplantation due to the time needed to mature into fully functional, insulin-secreting B-cells in vivo. This limitation holds true regardless of convention or vascularization status. Thus, we plan to continue exploring the possibilities of SCBC-loaded ceMEDs in subsequent long-term assessments.

As with most implanted biomaterials and devices, foreign body response (FBR) establishes around the ceMED within weeks after transplantation. The recruited macrophages fuse to form foreign body giant cell (FBGC) and coalesce with recruited fibroblasts and endothelial cells to deposit fibrotic tissue around the device. It was interesting to observe that the thickness of fibrosis on the surface of the ceMED was significantly decreased while convection was in place. It may be possible that the no flow group housed more apoptotic cells due to the lack of nutrient transfer and internal hypoxia. Thus, more apoptotic cell antigens and inflammatory signals may have traversed the device membrane and attracted phagocytes to the nearby tissue. Furthermore, as postulated by other studies exploring the effect of mechanical perturbation for reducing FBR, the mechanical movement of fluid flow may have decreased the FBR surrounding our device.

In this study, we chose to study fibrosis development after 14 days of transplantation, according to previous studies. Initially post-transplantation, FBR may not be as problematic as previously assumed because convection alone can facilitate ceMED functionality before vascularization. On the other hand, FBR may be a barrier over the long term once vascularization is replaced by extensive fibrosis. The progression of FBR may hinder glucose sensing and insulin secretion. Hence, in the future, we will investigate the mechanism by which flow reduces FBR and whether this is sustainable. We will also attempt to fully understand the series of immune events and effects that impact long-term device performance. Further studies to introduce flow as a general principle for reducing FBR in implants may also be desirable. To achieve long-term and self-mediated suppression of inflammation, immune activity, and fibrosis around the device, multiple strategies may be explored. For example, we may consider engineering ß cells or other accessory cells to secrete immunomodulatory factors and microfabricating membranes to achieve precise pore size control. Alternatively, using long-term controlled release of antifibrotic drugs may be explored in overcoming the challenges associated with FBR.

Overall, the enhanced cell viability and significant reduction in blood glucose with minimal delay following transplantation demonstrated for the ceMED, illuminates significant advantages over diffusion-based devices. Therefore, encapsulation coupled with the convection-aided design represents a viable approach to enhance the success of ß cell replacement therapies to treat T1D.

### COMSOL® Simulation

Prior to in vitro experimentation, to first predict the minimum flow rate required to maintain islet survival and function, we performed computational modeling using COMSOL®. The CC was assumed to be axisymmetric with respect to the HF flow channel to save computational power and assumes 100% equilibration of the fiber. The islets were distributed uniformly around the fiber in multilayers. The length of the system (both EqC and CC: 10 mm), HF diameter (inner diameter, 600 μm), HF wall thickness (100 μm) were based on the prototype design of the device. The diameter of islets was assumed as 150 μm and 4 layers of islets are housed between the semipermeable HF and PTFE membrane (double layered). The model accounts for the diffusion of nutrients through the porous membrane of the CC. It assumes oxygenated and glucose-rich environment outside the porous membrane and that the perfusate entering the HF has completely equilibrated with the environment. For detailed rationale and set up of COMSOL simulation, please see below.

### Cell Culture

SCβCs: The clusters at stage 6 day 15-25 were generously provided by Professor Melton (Harvard University) and maintained in 30 mL spinner flasks (a type of single use bioreactor, ABLE Biott, Tokyo, Japan) using established procedures.

Primary Human islet: Primary islets were obtained from Prodo Laboratories (Aliso Viejo, CA, USA) under guidelines approved by the Brigham and Women's Hospital Biosafety Registrations. Islets were suspended in PIM(T)™ media (Prodo Laboratory) upon arrival and immediately transferred into encapsulation device.

MIN6 cells: MIN6 cells (ATCC® CRL-11506™, Manassas, VA, USA) were plated in T-75 flasks and cultured with Dulbecco's Modified Eagle's Medium (DMEM, ATCC® 30-2002™) supplemented with 15% fetal bovine serum (FBS) (S11550, Atlanta Biologicals, Flowery Branch, GA, USA) and 2% Penicillin-Streptomycin (10,000 U/mL, Thermo Fisher Scientific, Waltham, MA, USA). Cells from low passage number (<5) were used. All cell culture was maintained in a humidified incubator with 5% CO₂ at 37° C.

### Device Fabrication

The design for the main structure of the ceMED was completed using graphical illustration software (CorelDRAW) and produced with laser-cutter on cast acrylic sheet-poly(methyl methacrylate) (McMaster-Carr, Aurora, OH, USA). In one embodiment, the device is 3.2 mm thick, 10 mm in length for CC and EqC, and 20 mm in length in total (dual EqC ceMED: 30 mm in length). CC is 150 μl in volume and a cell-seeding port is located on one side. Acrylic skeleton is attached with bilayer PTFE membranes (inner layer: 0.2 μm pore size, 85 μm thickness, from Sterlitech, Kent, WA, USA; outer layer: 10 μm pore size, 80% porosity, 85 μm thickness, from Millipore Sigma, Burlington, MA, USA) using acrylic solvent cement (Scigrip, Durham, NC, USA). A pre-carved gap in the center of ceMED houses the HF (modified polyethersulfone, MWCO 100 kDa, inner diameter 0.6 mm, from Repligen, Waltham, MA, USA) which is secured to the acrylic skeleton by epoxy glue (Loctite, Düsseldorf, Germany) and a connector at the entry point helps to connect to the silicone tubing (Tygon formulation 3350, Saint Gobain Performance Plastics, Courbevoie, France) and pump. Devices were sterilized by ethanol wetting and UV treatment for 2 hours followed by sterile PBS washes. The SCβCs, MIN6, and primary human islets were loaded into the ceMED and the loading port was sealed with Dermabond™ (Johnson & Johnson, New Brunswick, NJ, USA).

### In Vitro Glucose Equilibration

Devices with different lengths of EqC (5, 10, and 20 mm) were fabricated for in vitro testing of glucose equilibration. For static control group, an isolated CC was used. Empty ceMEDs without cell sample were connected with inflow and outflow silicone tubing and submerged into a 10 mL reservoir with 5 mM glucose dissolved in PBS. Inlet was continuously pumped with PBS at multiple flow rates (100, 250, 500, and 1,000 μl/hr). Small aliquots from the outflow or directly from the CC were collected for glucose concentration measurement at various time points with the Amplex™ Red Glucose assay (Thermo Fisher). To test responses to glucose dynamic changes, a ceMED was first submerged in a reservoir with 5 mM glucose concentration dissolved in PBS. After complete equilibration and saturation, fluid in the CC was collected through the cell loading port using a syringe with 30 gauge needle and dispensed in a centrifuge tube. A step change in glucose was stimulated by immersing the ceMED in a 13 mM glucose concentration reservoir then back into a 5 mM reservoir. Fluid in the CC was collected at multiple time points and measured by Amplex™ Red Glucose assay (Thermo Fisher).

### Optimization of Loading Capacity

Two-fold serial dilutions of SCβCs were performed to prepare cell densities of 20, 10, 5, and 2.5 IEQ/μL. SCβCs were then loaded into Matrigel (growth factor reduced, Corning, Corning, NY, USA) or alginate hydrogel (Millipore Sigma) embedded devices through the cell loading port and incubated in culture medium at 37° C. and 5% CO₂ either with perfused flow through the HF (100 μL/h) or no flow. Devices in the flow-enhanced condition were infused with PBS (Millipore Sigma) through silicone tubing (Saint Gobain Performance Plastics) connected at the entry point. Two days after seeding, viability of SCβCs was measured using alamarBlue assay (Invitrogen) and normalized to control cells cultured in spinner flask at respective densities. After optimization of cell density, cells at 10 IEQ/μL (150 μL) were loaded into CC for further experiments in vitro.

### Gene Expression Analysis

Quantitative real-time polymerase chain reaction (qRT-PCR) analysis was performed under a previously published protocol. MIN6 and SCβCs were loaded into sterilized ceMEDs at a density of 10 million cells/mL and 10 IEQ/mL (with a 150 UL in a device). After culturing, the devices were extracted, and the PTFE membranes were removed to expose cell samples. Total RNA was retrieved with the RNeasy Mini kit (Qiagen, Chatsworth, CA, USA) following manufacturer's instructions. The RNA concentrations were quantified by NanoDrop spectrophotometer (Thermo Fisher). Similar amounts of RNA contents from each sample were loaded for cDNA synthesis using QuantiTect reverse transcription kit (Qiagen) following manufacturer's instructions. qRT-PCR was performed with 7900 Fast Real-Time PCR System (Applied Biosystems, Foster City, CA, USA). SYBR™ Green PCR master mix (Applied Biosystems) and the following QuantiTect Primer Assays (Qiagen), with respective Entrez Gene ID, were used: mouse BCL2 (12043), human BCL2 (596), mouse BAX (12028), human BAX (581), mouse glyceraldehyde 3-phosphate dehydrogenase (GAPDH) (14433), human GAPDH (2597). Target gene expression levels were analyzed by the comparative Ct method and represented as relative comparison to the static control group after normalization to endogenous GAPDH content.

### Immunocytochemistry and Immunohistochemistry for Retrieved Devices

Retrieved devices were fixed by submersion in 4% paraformaldehyde (Electron Microscopy Sciences) for 30 minutes at 4° C. and washed once in PBS. Devices were then put onto a platform for excision of the content in the CC using a surgical blade while keeping the samples frozen by surrounding dry ice. Excised samples were embedded in optimal cutting temperature (OCT) compounds (Sakura Finetek, Torrance, CA, USA), and the blocks were allowed to harden on dry ice before being processed for cryosection. Cryosectioned slides were blocked with 5% donkey serum (Jackson ImmunoResearch, West Grove, PA, USA) for 30 minutes at 4° C. and washed twice in PBS. For devices used for in vitro study, the TUNEL assay (Invitrogen) was used to identify apoptotic cells following manufacturer's instructions. Cellular nuclei were backstained with Hoechst 33342 (Invitrogen). For devices extracted from in vivo studies, the slides were immersed in primary antibody mixture overnight at 4° C. The following primary antibodies were used at the indicated dilution factor: mouse monoclonal anti-C-peptide (1:300, Cell Signaling Technology, Danvers, MA, USA), rabbit monoclonal anti-Glucagon (1:1,000, Abcam, Cambridge, UK), goat polyclonal anti-somatostatin (1:500, Santa Cruz, Dallas, TX, US), rabbit polyclonal anti-Coll (1:500, Abcam), mouse monoclonal anti-SMCa (1:500, Millipore Sigma), mouse monoclonal anti-CD31 (1:500, Abcam), mouse monoclonal anti-CD68 (1:500, Abcam). Samples were then washed thrice in PBS and stained with secondary antibodies, prepared in block solution at 1:500 dilution factor, for 30 minutes at 4° C., then washed thrice in PBS. The following secondary antibodies were used: donkey anti-rabbit Alexa Fluor-647 (Invitrogen), goat anti-mouse Alexa Fluor-488 (Invitrogen), goat anti-mouse Alexa Fluor-405 (Invitrogen). Nuclear staining was performed with 4′,6-diamidino-2-phenylindole (DAPI, Millipore Sigma).

### GSIS

The analysis was conducted following previously described procedures. The ceMEDs were loaded 150 μl of MIN6 cells or SCβCs at a seeding density with 3 million cells or 2,000 IEQ. For SCβCs, the cells were extracted from rat subcutaneous space 14 days post-transplantation and then measured using GSIS analysis. After overnight culture, the devices were removed from culture media and starved in 1.5 mL 2.8 mM glucose (Millipore Sigma) solution for 2 hours. The devices were then sequentially submerged in solutions of 2.8 mM glucose, 20 mM glucose, 2.8 mM glucose, and 30 mM KCl for either 30 or 60 minutes each. Small aliquots of the solutions (10 μl) were collected at each timepoint and insulin content was quantified by mouse insulin ELISA (Mercodia, Uppsala, Sweden) and Human Ultrasensitive Insulin ELISA kit (ALPCO Diagnostics, Salem, NH, USA). Then, we collected and dispersed the cells with TrypIE (15 min), stained them with Trypan blue, and normalized to the number of live cells.

### Device Implantation

All animal experiments were performed under the approved Institutional Animal Care and Use Committee (IACUC) protocol by the Center of Comparative Medicine (CCM) at Brigham and Women's Hospital at Harvard Medical School. Immunocompetent Lewis Rat (LEW/Crl), 10˜12 weeks old, were purchased from Charles River Laboratory (MA, USA) and used as recipients. Animals were anesthetized with vaporized isoflurane in oxygen (3% for induction, 1% for maintenance). Areas on the dorsal region were shaved and prepared with povidone-iodine and ethanol wipes. Animals were given preemptive analgesics in a single dose of 5 mg/kg Meloxicam (Patterson Veterinary, Greeley, CO, USA) delivered subcutaneously. An incision less than 2 cm was made along the midline of the prepared dorsal region to create an opening into the subcutaneous space and a pair of blunt-end scissors was used to create a subcutaneous pocket for the device. A small incision was made at the other end of the pocket to act as the exit port. The devices (seeded with cells in the CC) were then removed from sterile containers, threaded through the opening, and placed into the subcutaneous region using two pairs of tweezers. The inlet and outlet silicone tubing were placed through the two incisions and connected to extension tubing sterilized by ethanol wetting and PBS wash. The incision sites were closed using 3-0 non-absorbable Nylon sutures with reverse-cutting needle (Ethicon, Somerville, NJ, USA) which were removed after 14 days. Sutured skins were cleansed with ethanol wipes and applied with antibacterial ointment prior to putting on the tethered harness for swivel cage set-up.

### Swivel Cage

To enable unobstructed infusion into the implanted devices and to prevent interference from animal movements, each animal was singly housed, under the approval of IACUC and CCM, and tethered under the swivel cage set-up. This setting allows us to conveniently implant the device subcutaneously while pumping fluid through and collecting equilibrated fluid outside for analysis and therefore, monitor the glucose content of the fluid exiting the HF. The tethered system included a harness, a spring tether, a two-channel swivel, a swivel mount, extension tubing which were all purchased from Instech Laboratories (Plymouth Meeting, PA, USA), and a syringe pump (NE-1600, New Era Pump Systems, Farmingdale, NY, USA). The adjustable harness, worn around the forelimbs of the animal, is connected to the stainless steel spring tether which protects the device tubing and transmits rotary movement to the swivel which was clamped onto the counter-balanced swivel mount positioned on top of the rat cage. The extension tubing was connected to the syringe pump.

### In Vivo Glucose Equilibration

For in vivo testing of glucose equilibration across the exposed HF in ceMED, an isolated EqC was fabricated, sterilized, and transplanted into non-diabetic Lewis rats under the protocol outlined above. The inlet was pumped with either 2 mM or 20 mM sterilized glucose solution (PBS) at multiple flow rates (250, 500, and 1,000 μl/hr). The outflow exiting the subcutaneous space and tail vein blood was collected at various time points. Small aliquots extracted from the outflow samples were analyzed for glucose concentration with Amplex™ Red Glucose assay (Thermo Fisher). The blood glucose level was measured using a glucose meter (Accu-Chek® Guide, Roche Diabetes Care, Mannheim, Germany).

In Vivo Glucose Monitoring after Device Transplantation

Sterilized ceMEDs were loaded with primary human islets or MIN6 at 2,000 IEQ (3×10⁶ cells) and transplanted into the subcutaneous space of animal recipients. For this study, immunocompetent Lewis rats were induced to become diabetic through a single IP injection of 60 mg/kg STZ suspended in PBS. The rats were chosen for transplantation if next-day blood glucose increased to above 300 mg/dl. Blood was collected from the tail vein every 2˜5 days to assess the glucose concentration using a glucose meter (Accu-Chek®).

### Intraperitoneal Glucose Tolerance Test

At day 14, intraperitoneal glucose tolerance test (IPGTT) was performed. The rats were starved for 12 hours and then injected with 2 g/kg glucose suspended in PBS through IP. Tail vein blood was collected just prior to injection and at various time points after injection for reading with glucose meter (Accu-Chek® Guide, Roche Diabetes Care). The human insulin level was measured using collected blood by human insulin ELISA kits (Alpco, Salem, NH, USA), respectively, until device extraction.

Retrieval of ceMED and Processing

Devices were retrieved after 14 days on the basis of other transplantation experiments and fibrotic encapsulation studies. After the animals were euthanized with CO₂, the devices were extracted from the dorsal subcutaneous space and washed twice in PBS. Retrieved devices were fixed, cryosectioned, and processed for histological sectioning. Histological sections were then stained with Hematoxylin and Eosin (H&E) and Masson's trichrome staining at the Koch Institute at MIT or processed for immunohistochemical imaging. Quantification of thickness of fibrosis was performed on H&E stained sections using ImageJ software (NIH). To visualize potential angiogenesis, the surface of device was stained with 3,3′-Diaminobenzidine (DAB) with horseradish peroxidase (HRP) substrate (Invitrogen) and imaged with a bright-field microscope. To retrieve the encapsulated cells for insulin secretion test, the devices were washed with PBS thoroughly and the PTFE membrane on one side of the CC was lifted off using a tweezer. The enclosed cells were extracted and transferred to a well plate. The cells were tested for insulin secretion following 14 days by ELISA kit (R&D systems, Minneapolis, MN, US). The GSIS was performed as previously described in the GSIS section. For immunofluorescent staining of endocrine markers (C-peptide, Glucagon and Somatostatin), cells were extracted from the CC following 7 days post-transplantation, and staining was performed as described above.

### Statistical Analysis

All experimental data were analyzed with GraphPad Prism v8 (GraphPad Software, San Diego, CA, USA). Values were presented as mean ± standard deviation and assessed for statistical significance using one-way or two way analysis of variance (ANOVA) followed by multiple comparison tests (Dunnett's or Tukey's) where * indicates P<0.05, ** indicates P<0.01, and *** indicates P<0.001. The displayed in vitro data was collected from at least 3 biological replicates. In vivo validation was also collected from 2˜3 biological replicates. When comparisons to multiple groups were made, additional symbols were used. Number of replicates were indicated under the figure captions for each graph.

### Supplementary Information

COMSOL® Simulation: Modeling of Nutrient Transport in Static Device and ceMED

The pancreatic islets mainly contain the endocrine cells (a, B, y, and pancreatic polypeptide-cells) and their main role is to secrete hormones to maintain the blood glucose level in the body. These islets need a constant supply of nutrients and oxygen to generate insulin. To demonstrate the nutrient transport limitations of the TheraCyte™, we modeled a static macroencapsulation device (MED) and compared its performance in silico to convection-enhanced MED (ceMED). The coupled mass transport equations for glucose, oxygen, and insulin were solved for islets within versions of these devices, modelled in 2D using COMSOL 5.0 multiphysics. In this model, islets are assumed to be perfect spheres that are packed within the capsule. To modularly demonstrate the effects of capsule expansion, the interior is divided into 150 μm-thick islet “layers”. In the present model, the encapsulated islets are assumed to consume the nutrient, glucose, and oxygen. They also act as insulin source for both local glucose concentrations and time variation of the glucose concentrations. In the traditional TheraCyte™-type device, nutrient and oxygen transport to islets take place due to diffusion only, which is a very slow transport process. This slow transport from the periphery of the TheraCyte™ limits the oxygen supply to the inner core of the islets, which leads to hypoxia, loss of functionality, and cell death. Thus, it is necessary to understand the mass transport of oxygen, glucose, and nutrients to the islets in the TheraCyte™. The current mathematical model focuses on these issues for effective transport of oxygen, glucose, and nutrients to islets and insulin release rate out of TheraCyte™. It has been reported that insulin release by islets follows a biphasic behavior. Here, a square wave pulse of glucose elicits a two-phase release of insulin. The first phase of insulin release (a transient spike) is followed by a slower second phase. This insulin release rate also depends on oxygen availability and decreases nonlinearly with decreasing oxygen concentration. Accordingly, it is necessary to incorporate this biphasic nature of insulin release in this model.

### Modeling

In the present model, the islets are modeled to secrete the insulin in response to the local glucose concentration level (c_{g}) as well as the glucose concentration-time gradient (ξc_{g}/ξt). The typical insulin release profile follows a Hill-type sigmoid response as a function of glucose concentration change in the first phase and local glucose concentration in the second phase. As the functionality of the islets depends on nutrients, glucose, and oxygen, the glucose consumption is also incorporated into the model using the Michaelis-Menten type kinetics. The availability of oxygen is a limiting factor to maintain islet insulin secretion in response to glucose. In addition, islet death due to hypoxia is another factor. Therefore, a critical oxygen concentration affects cell function and cell viability. In the present model, a total of three concentrations for glucose, oxygen, and insulin respectively (c_{g}, cₒ, cᵢₙ) are used with their corresponding mass transfer equation. In the absence of convective flow, the mass transfer is taking place due to diffusion only. The equation of mass transport equation is given as:

$$\frac{\partial {c}_{i}}{\partial t} + \vec{u} \cdot ∇ {c}_{i} = {D}_{i} {∇}^{2} {c}_{i} + {R}_{i}$$

Where, cᵢ denotes the concentration [mol m-3], Dᵢ is the diffusivity constant [m²s⁻¹], Rᵢ the consumption/release reaction rate [mol m⁻³s⁻¹], i denotes the parameter for glucose, oxygen and insulin in the system, u represents the velocity field [m²s⁻¹] for convection, and ∇ is the standard del operator for the Cartesian coordinate system. In the present study, the two different diffusion coefficients for aqueous media (D_{i.aq}) and islet tissue (D_{i,t}) with their respective species are used (Table 1). The consumption/release rates in the mass transport equation were assumed to follow Hill-type dependence on their respective concentrations and represented by the generalized Michaelis-Menten kinetics:

$${R}_{i} = {R}_{max} \frac{{c}_{i}^{n}}{{c}_{i}^{n} + {C}_{Hf}^{n}}$$

Where, Rₘₐₓ denotes the maximum consumption/release rate, C_{Hf}ⁿ concentration corresponds to half of the Rₘₐₓ, and n slope constant characterizes the shape of response. The glucose consumption rate for islet is modeled the same Michaelis-Menten kinetics with the respective parameters for glucose.

$${R}_{g} = {R}_{g , max} \frac{{c}_{g}^{{n}_{g}}}{{c}_{g}^{{n}_{g}} + {C}_{Hf , g}^{{n}_{g}}}$$

On the other hand, the oxygen consumption rate for islet is modeled using Michaelis-Menten kinetics with the incorporation of two more parameters (φ_{o,g}, δ). The first parameter (φ_{o,g}) accounts for the increased metabolic demand of oxygen for higher glucose concentrations. The second parameter (δ) accounts for cell death due to hypoxia and represents the condition where oxygen concentration falls below the critical oxygen concentration (c_{o,cr}) required by the islet to survive.

$${R}_{o} = {R}_{o , max} \frac{{c}_{o}^{{n}_{o}}}{{c}_{o}^{{n}_{o}} + {C}_{Hf , o}^{{n}_{o}}} \cdot {φ}_{0 , g} \cdot δ ( {c}_{o} > {c}_{o , cr} )$$

While the (δ) is a step-down function, depending on the local oxygen concentration, the (φ_{o,e}) increases with the metabolic demand along with insulin secretion rate as a function of the glucose concentration and given as:

$${φ}_{o , g} = {φ}_{sc} ( {φ}_{base} + {φ}_{meta} \frac{{c}_{g}^{{n}_{i 2 , g}}}{{c}_{g}^{{n}_{i 2 , g}} + {C}_{Hf , in 2 , g}^{{n}_{i 2 , g}}} )$$

The value of (φ_{sc}, φ_{base}, φₘₑₜₐ) are used to calculate the increased metabolic demand.

The insulin secretion rate depends on the local glucose and oxygen concentrations. To capture the biphasic behavior of the insulin secretion for a step input glucose response, the insulin release is modeled as first phase (R_{in,Ph1}) and second phase (R_{in,Ph2}) release rates as given below:

$${R}_{in , ph 1} = {R}_{in , max , Ph 1} \frac{{( \frac{\partial {c}_{g}}{\partial t} )}^{{n}_{Ph 1}}}{{( \frac{\partial {c}_{g}}{\partial t} )}^{{n}_{Ph 1}} + {C}_{Hf , g , Ph 1}^{{n}_{Ph 1}}} \cdot ( \frac{4 {c}_{g}^{4} {c}_{m}^{4}}{{( {c}_{g}^{4} + {c}_{m}^{4} )}^{2}} )$$

$${R}_{in , ph 2} = {R}_{in , max , Ph 2} \frac{{c}_{g}^{{n}_{Ph 2}}}{{c}_{g}^{{n}_{Ph 2}} + {C}_{Hf , g , Ph 2}^{{n}_{Ph 2}}}$$

While the insulin released during one glucose cycle is given as sum of the first and second phase release, the insulin release (R_{in,Ph1}+R_{in,Ph2}) is multiplied by a modulating factor to limit the insulin release for a local oxygen concentration that is below ˜6 HM.

$${R}_{i n} = ( {R}_{in , Ph 1} + {R}_{in , Ph 2} ) \cdot ( \frac{{c}_{o}^{3}}{{c}_{o}^{3} + {C}_{Hf , o , in}^{3}} )$$

Furthermore, to match the correct time scale of the insulin release, a local compartment is added to facilitate the sustained release to a glucose response by following first order kinetics.

$$\frac{d {c}_{inL}}{d t} = {R}_{in} - {k}_{inL} ( {c}_{inL} - {c}_{in} )$$

The velocity field u for the convection is calculated using the continuity and Navier-Stokes equations for Newtonian incompressible fluid.

$$∇ \cdot \vec{u} = 0$$

$$p ( \frac{\partial \vec{u}}{\partial t} + \vec{u} \cdot ∇ \vec{u} ) = - ∇ p + μ {∇}^{2} \vec{u}$$

Here, p denotes density [kg m⁻³], u viscosity [kg m⁻¹s⁻¹], and p pressure [kg m s⁻²]. In the present study, the body force term is omitted in the Navier-Stokes due to small size of the device.

### Geometry and Meshing

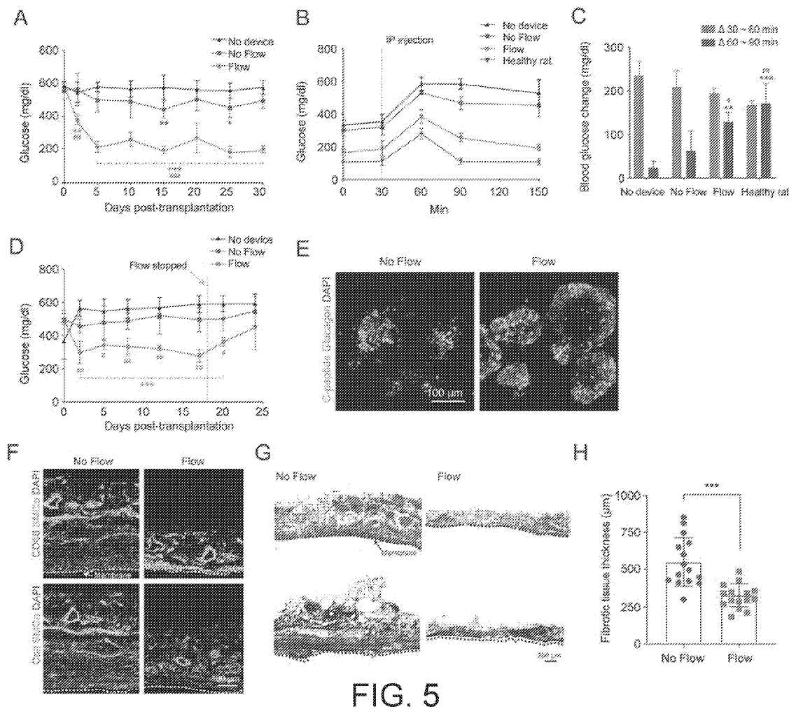

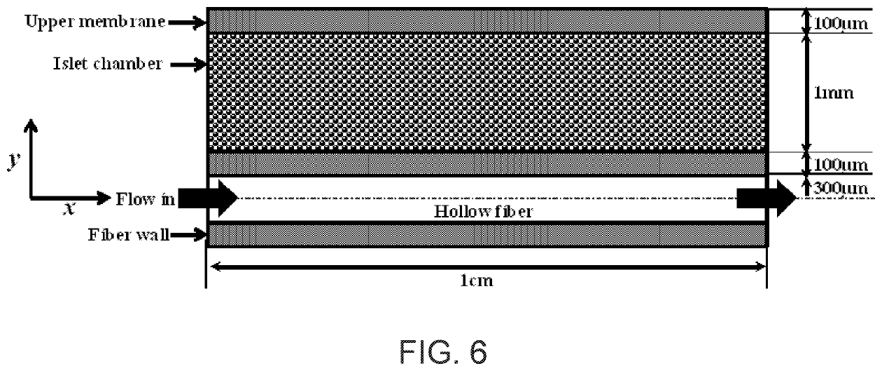

The present device is modeled as a 2D device to save on computation cost and model efficiency. A 2D geometry is created with spherical islets of 150 μm diameters placed inside it. The device dimensions are given in the scheme shown in FIG. 6. For meshing, COMSOL's default “extra fine mesh” option was used to generate the device mesh with 90,000˜110,000 elements and minimum element size of 1.2 μm.

### Parameter Settings

The parameters used to model the convection flow, islet's nutrient consumption, and insulin secretion rates with their respective units are provided in Table 1 below. Although silicone tubing is used during perfusion due to robust oxygen diffusion characteristics, they were not included in the islet model as it is not the part of the CC.

TABLE 1

| Parameter | Value | |
|---|---|---|
| Fiber inner radius | 300e−6 | [m] |
| Fiber membrane thickness | 100e−6 | [m] |
| Fiber length | 0.01 | [m] |
| Cell “Islet” chamber height | 2e−3 | [m] |
| Cell “Islet” chamber length | 0.01 | [m] |
| Inlet velocity (Vₘₐₓ) | 0.0001 | [m/s] |
| Inlet glucose concentration (C_{g, inlet}) | 1.0 | [mol/m³] |
| Inlet oxygen concentration (C_{o, inlet}) | 0.2 | [mol/m³] |
| Inlet insulin concentration (C_{in, inlet}) | 0.0 | [mol/m³] |
| Diffusivity of glucose in the aqueous media | 9e−10 | [m²/s] |
| (D_{g, aq}) | | |
| Diffusivity of glucose in the islet tissue (D_{g, t}) | 3e−10 | [m²/s] |
| Diffusivity of oxygen in the aqueous media | 3e−9 | [m²/s] |
| (D_{o, aq}) | | |
| Diffusivity of oxygen in the islet tissue (D_{o, t}) | 2e−9 | [m²/s] |
| Diffusivity of insulin in the aqueous media | 1.5e−10 | [m²/s] |
| (D_{in, aq}) | | |
| Diffusivity of insulin in the islet tissue (D_{in, t}) | 0.5e−10 | [m²/s] |
| Glucose consumption rate (R_{g, max}) | −0.028 | [mol m⁻³ s⁻¹] |
| Oxygen consumption rate (R_{o, max}) | −0.034 | [mol m⁻³ s⁻¹] |
| Insulin secretion rate (R_{in, max, Ph1}) | 21e−5 | [mol m⁻³ s⁻¹] |
| Insulin secretion rate (R_{in, max, Ph2}) | 3e−5 | [mol m⁻³ s⁻¹] |
| Glucose Michaelis-Menten constant (C_{Hf, g}) | 10e−3 | [mol m⁻³] |
| Oxygen Michaelis-Menten constant (C_{Hf, o}) | 1e−3 | [mol m⁻³] |
| Insulin Michaelis-Menten constant (C_{Hf, g, Ph1}) | 0.03 | [mol m⁻³ s⁻¹] |
| Insulin Michaelis-Menten constant (C_{Hf, g, Ph2}) | 7.0 | [mol m⁻³] |
| C_{Hf, o, in} | 3e−3 | [mol m⁻³] |
| C_{o, cr} | 1.0e−4 | [mol m⁻³] |
| φ_{sc} | 1.8 | |
| φ_{base} | 0.5 | |
| φₘₑₜₐ | 0.5 | |
| Cₘ | 5.0 | [mol m⁻³] |
| k_{inL} | 0.003 | [s⁻¹] |
| n_{Ph1} | 2.0 | |
| n_{Ph2} | 2.5 | |
| n_{i2, g} | 2.5 | |
| nₒ | 1 | |
| n_{g} | 1 | |
| Density of aqueous media (ρ) | 998 | kg m⁻³ |
| Viscosity of aqueous media (μ) | 10e−3 | Pa · s |

Table S. Parameters used in the present model.

### Boundary Conditions

The convection enhanced insulin secretion model is solved using COMSOL metaphysics using flow Free and Porous Media Flow and Transport of dilute species modules. The velocity field is modeled using Navier-Stokes equation for incompressible flow in porous media. The glucose, oxygen, and insulin concentration field are modeled using transport of dilute species modules with convection transport mechanism. The coupled model is implemented in COMSOL 5.0 and solved as time-dependent (transient) problems using PARDISO direct solver for time step of 1.0 s. The simulation was run for 1000 s in order to achieve the steady-state. The following boundary conditions were used for this Navier-Stokes model: the parabolic inflow velocity profile boundary condition at the fiber inlet, a zero pressure boundary condition is used at both fiber outlets as well as at the outside membrane; all other solid walls are described by a no-slip boundary condition. The geometry is assumed to be symmetric along the axis of the fiber, and symmetric velocity boundary condition is used at the center of fiber. For the transport of dilute species modules, the no flux boundary conditions n (−D∇c+cu)=0 are used for device walls. The inflow for glucose and oxygen concentrations boundary condition are used (C_{g.inlet}=1 mol/m³, C_{o.inlet}=0.2 mol/m³, C_{in.inlet}=0.0 mol/m³). While the insulin concentration was taken as zero at inlet, zero convective flux was used for glucose, oxygen, and insulin at the outlet. The initial boundary condition for glucose and oxygen concentrations used are C_{g.int}=6.0 mol/m³, C_{o.int}=0.2 mol/m³, C_{in.int}=0.0 mol/m³.

### Simulations

Simulation of glucose transport into the capsule from the membrane surface revealed that islets immediately adjacent to the membrane receive only 75˜90% of the concentration of interstitial glucose. The amount of glucose reaching the adjacent islet layers decreases due to metabolic consumption by inner layers of islets with the thickness of the chamber. Consequently, even islets within optimal static MEDs do not receive the interstitial concentrations of nutrients, which may negatively impact their ability to physiologically respond to blood glucose levels. Simulation of oxygen transport revealed hypoxic conditions, even for those islets situated at the membrane surfaces (<25% of interstitial oxygen concentration). If the device chamber is increased in size, interior islet layers receive <0.0001 mM of oxygen, resulting in decreased function and cell death. Both glucose and oxygen concentrations influence the amount of insulin secretion by encapsulated islets. The static model demonstrates negligible insulin secretion by any islets not situated adjacent to the membrane surface.

A Closed-Loop Recirculation ceMED System for Long-Term Treatment

To eliminate the need for an external pump or the combination of an internal pump with a reservoir that needs periodic replenishment, we performed a benchtop validation of the use of a peristaltic pump and rewiring of the fluidic circuit to enable recirculatory convective flow through the CC. Some one-way implantable pumps (e.g., Medtronics's Synchromed II™ and Flowonix's Prometra II™) are available for use and should allow the implantation of the device subcutaneously. However, limitations of a one-way system include the need for a fluid reservoir which require frequent refills at high flow rates and the formation of edema as a result of the imbalanced absorption rate of interstitium to fluid flow rate. Based on the challenges associated with a one-way system, we seek to develop a closed loop recirculation system using a recirculating pump (e.g., peristaltic pump) considering the long-term application of the ceMED (See below, FIG. 11A). To test such a system, we placed the EqC and CC in two separate reservoirs with 10 mL with a FITC-dextran (4 kDa) (5 mM) (See below, FIG. 11B). Then, the fluid from the CC was collected to investigate its concentration using a microplate reader. Excitation and emission wavelength of 490 nm and 525 nm were used. After 15 minutes, the diffusion-based device (No flow) had 44.9+3.8% equilibration, whereas the perfusion group with peristaltic pump showed 92.8+6.5% equilibration (See below, FIG. 11C). This result indicates that the ceMED with a closed loop system can afford equilibration with the surrounding solution.

While the various embodiments disclosed herein may specify use of certain materials, those skilled in the art will understand that other materials may be used, as disclosed below.

### Device Scaffold:

Various polymers and polymer blends can be used to manufacture the device jacket, including, but not limited to, polyacrylates (including acrylic copolymers), polyvinylidenes, polyvinyl chloride copolymers, polyurethanes, polystyrenes, polyamides, cellulose acetates, cellulose nitrates, polysulfones (including polyether Sulfones), poly phosphaZenes, polyacrylonitriles, poly(acrylonitrile/covinyl chloride), PTFE, as well as derivatives, polyethylene and polyetheylene-derived membranes (e.g., PET), copolymers and mixtures of the foregoing. Various polymers such as described in U.S. Pat. No. 9,526,880, incorporated herein by reference in its entirety, are possible.

### Device Semi Permeable Membrane:

Preferred devices may have certain characteristics which are desirable but are not limited to one or a combination of the following: i) including a biocompatible material that functions under physiologic conditions, including pH and temperature; examples include, but are not limited to, anisotropic materials, polysulfone (P SF), nano-fiber mats, polyimide, tetrafluoroethylene/polytetrafluoroethylene (PTFE; also known as Teflon®), ePTFE (expanded polytetrafluoroethylene), polyacrylonitrile, polyethersulfone, acrylic resin, cellulose acetate, cellulose nitrate, polyamide, graphene and graphene derivatives, as well as hydroxylpropyl methyl cellulose (HPMC) mem-branes; ii) releases no toxic compounds harming the biologically active agent and/or cells encapsulated inside the device; iii) promotes secretion or release of a biologically active agent or macromolecule across the device; iv) promotes rapid kinetics of macromolecule diffusion; v) promotes long-term stability of the encapsulated cells; vi) promotes vascularization; vii) including membranes or housing structure that is chemically inert; viii) provides stable mechanical properties; ix) maintains structure/housing integrity (e.g., prevents unintended leakage of toxic or harmful agents and/or cells); x) is refillable and/or flushable; xi) is mechanically expandable, as disclosed in US 2011/0280915, incorporated herein by reference in its entirety.

### Hollow Fiber:

Biocompatible semi-permeable hollow fiber membranes, and methods of making them are disclosed in U.S. Pat. Nos. 5,284,761 and 5,158,881 (see also, WO 95/05452), each of which is incorporated herein by reference in its entirety; see also U.S. Pat. No. 9,526,880, incorporated herein by reference in its entirety. In one embodiment, the device jacket is formed from a polyether sulfone hollow fiber, such as those described in U.S. Pat. Nos. 4,976,859 and 4,968,733, each incorporated herein by reference in its entirety.

In one embodiment, the encapsulating devices include a biocompatible material including, but are not limited to, anisotropic materials, polysulfone (PSF), nanofiber mats, polyimide, tetrafluoroethylene/polytetrafluoroethylene (PTFE; also known as Teflon®), ePTFE (expanded polytetrafluoroethylene), polyacrylonitrile, polyethersulfone, acrylic resin, cellulose acetate, cellulose nitrate, polyamide, polyethylene and polyetheylene-derived membranes (e.g., PET), as well as hydroxylpropyl methyl cellulose (HPMC) membranes. These and substantially similar membrane types and components are manufactured by at least Gore®, Phillips Scientific®, Zeus®, Pall® and Dewal®, among others.

### Hydrogel Material/Biocompatible Viscous Solution:

Additional materials that can be used for the hydrogel include: polyethylene-imine and dextran sulfate, poly(vinylsiloxane) ecopolymerepoly-ethyleneimine, phosphorylcholine, poly (ethylene glycol), poly(lactic-glycolic acid), poly(lactic acid), polyhydroxyvalerte and copolymers, polyhydroxybutyrate and copolymers, polydiaxanone, polyanhydrides, poly (amino acids), poly(orthoesters), polyesters, collagen, gelatin, cellulose polymers, chitosans, alginates, fibronectin, extracellular matrix proteins, vinculin, agar, agarose, hyaluronic acid, matrigel and combinations thereof. See U.S. Pat. No. 10,207,026, which is incorporated herein by reference in its entirety.

In various embodiments the hydrogel may include alginate, where the alginate has a concentration of guluronic acid of between 30% and 50%; in other embodiments the concentration of guluronic acid may be between 40% and 47%; in still other embodiments the alginate has a dry matter content of at least 1.6%; in yet other embodiments the alginate has a dry matter content of at least 2.1%; and in still other embodiments the alginate may be cross-linked with strontium. See US 2017/0157294, which is incorporated herein by reference in its entirety.

### Folded Implant Structures

The “Implant Area (IA)” refers to the 2D area of blunt dissection required to implant a device (or, put differently, the 2D area under the skin occupied by a device). IA is important for assessing the surgical invasiveness/complexity of device insertion, as well as determining the practicality of an implantable device.

In general, having larger blunt dissection area is more surgically invasive, dangerous, and painful to the patient. After recovery from surgery, rigid implants must not create discomfort as underlying tissues move. Ideally, these implants also do not create discomfort during daily activities, nor will everyday movements or positions damage the device. These may be issues that arise with certain known human-sized implant devices, which may confer a certain disadvantages such as a particular level of surgical risk, no candidate subcutaneous locations large enough to accommodate rigid/flat device, and a potential for everyday activities to bend, crush, or otherwise damage the device, etc. Given conventional technology (abbreviated ‘TC’ herein), it does not appear that the overall surface area of a macroencapsulation device relying on passive diffusion could be further optimized, which is fixed near 200-400 cm², and when presented as a flat device provides numerous obstacles to successful implantation, particularly over extended periods of time.

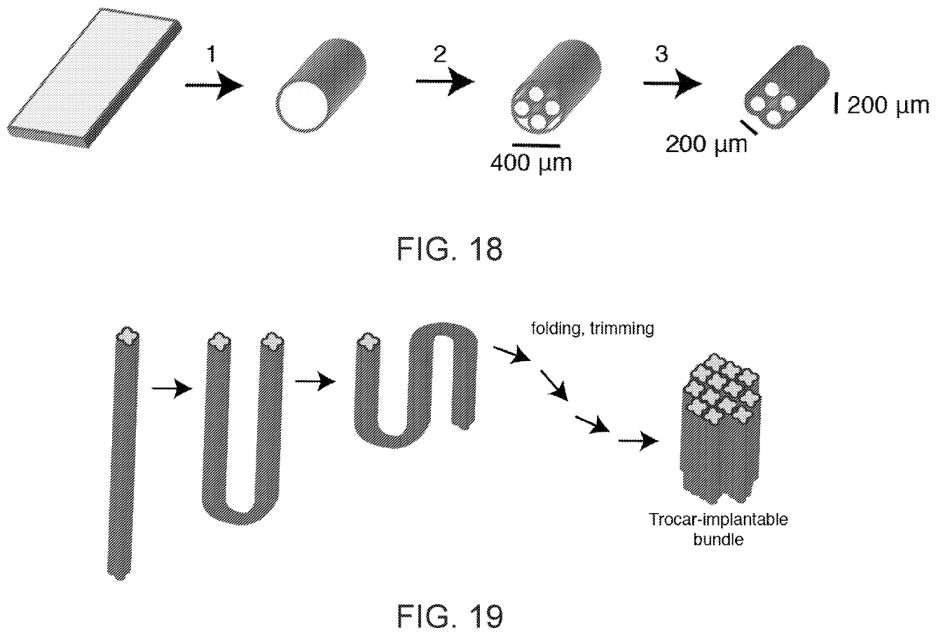

However, a passive diffusion device does not need to be flat or rigid. Thus, in certain embodiments, we provide implantable devices with substantial amounts of surface area that achieve a small IA through folding, which are sometimes referred to herein as “origami” structures. This principle is shown diagrammatically in FIG. 18.

An initial strategy that was considered is to fold a flat implant device into a tubular structure. See arrow 1 in FIG. 18. However, it is noted that vascularization of the interior of this tube structure would take longer than vascularization of more accessible surfaces. Consequently, we eliminate the hollow lumen to create a cylinder of packed tissues. FIG. 18, arrow 2. For optimal survival, this tube should have diameter no greater than 400 μm so that no encapsulated cells are beyond the 200 μm diffusion distance from the membrane. Additionally, the tubular surface membranes should have substantially similar properties to those of the original flat implant device to afford vascularization and immunoprotection. This tubular structure is further optimized by generating a more conformal structure (step indicated by arrow 3), in which cell “cubbies” in the tube walls ensure maximal surface area contact with the encapsulated tissue. These “cubby folds” are analogous to folded structures within the tissues such as the gastrointestinal system, which increase the surface area of the shape without increasing its occupying significant volume. We call this geometry a tubular rosette. The tubular rosette can be further folded into bundles to afford a compact, 3D geometry that is amenable to trocar-implantation (FIG. 19), which is referred to herein as the “Origami Macroencapsulation Device (MED).”

TABLE 2
The tubular rosette is modeled by extension
of rosette cross-sections.

| Parameter | Assumptions |
|---|---|
| Device | Model Tubular Rosette is optimal: |
| construction | Uniform packing of cells |
| | Fixed distance between membranes |
| | Uniform islet size and shape |
| | No deadspace in membrane (aporous regions) |
| Physical | Diffusion limit of tissue-sustaining |
| assumptions | nutrients = 200 μm |
| | Uniform prevascularization |
| | All tissues within 200 μm of membrane |
| | surfaces are functional |
| | All islets are 200 μm diameter spheres |
| | Islets packed in matrix (square packing in long |
| | axis, rosette packing in cross-section) |
| | Membrane thickness = 50 μm |
| | Rosettes are packed as cylinders², which is |
| | conservative (i.e., rosettes will pack more |
| | tightly than cylinders) |
| Known | # of islets to cure T1D rat with TC = 1k-2k |
| parameters | Rat-curing 4.5 μL TC (for comparison): |
| | Implant area (IA) = 0.4 cm² |
| | Surface area (SA) for nutrient exposure = 0.8 cm² |
| | Volume = 4.5 μL = 4.5 mm³ |
| | Geometry = rectangular prism |
| | Optimal islet capacity = 2000 |
| | # of islets needed to cure T1D human |
| | adult = 500k-1 million |

### Mathematical Modeling of the Origami Macroencapsulation Device

### One-Petal Tubular Rosette

The simplest cross section is a cylinder of inner diameter 0.2-0.25 mm, which can accommodate one islet in cross section. Because the islet is within 200 μm distance of the membrane along its circumference, this islet is assumed to be alive. Thus, such geometry holds 1 viable islet/cross-section and 5 viable islets/mm length.

To find the dimensions of a one-petal tubular rosette equivalent of the 4.5 μL TC, we determine the length of a 2000-islet one-petal tubular rosette:

Tubular Rosette Length=2000 islets/device * 1 mm length/5 islets=400 mm=40 cm

Thus, a 40 cm tubular rosette of singly-packed islets would be equivalent to the 4.5 μL TC. This rope can be further folded by bundling it into a larger, cylindrical shape. Recall the outer diameter of the rosette is the inner diameter+100 μm (thickness is 50 μm). Thus, assuming a 0.269 cm trocar diameter (10G needle), the number of 0.45 mm diameter tubes that can be packed is 43(3), and thus the total length of a 0.269 cm-diameter TC-comparable device is:
Device Length=Total Tube Length/Number of Tubes Packed into Trocar
Device Length (for 10G trocar)=40 cm/43=0.93 cm

Table 3 shows optimization of tubular rosette folding of a conventional technology (TC) device for reducing Implant Area in a rodent. Tubular rosettes can be bundled to afford trocar-implantable devices. A 4-petal rosette affords trocar-implantable bundles with the smallest Implant Area.

TABLE 3

| | | | | | 4.5 μL TC equivalent, 0.269 | | | |
|---|---|---|---|---|---|---|---|---|
| | | | | | cm diameter trocar, 2000 islets | | | |
| | | | islets | | length/ | | | |
| | | | (in x- | islets/ | 2000 | | device | |
| # of | OD | ID | sec- | mm | islets | # | length | IA |
| petals | (mm) | (mm) | tion) | length | (mm) | rods** | (cm) | (cm²) |
| 1 | 0.35 | 0.25 | 1 | 5 | 400 | 43 | 0.93 | 0.25 |
| 2 | 0.5 | 0.4 | 2 | 10 | 200 | 21 | 0.95 | 0.26 |
| 4 | 0.6 | 0.5 | 4 | 20 | 100 | 14 | 0.71 | 0.19 |
| 5 | 0.7 | 0.6 | 5 | 25 | 80 | 9 | 0.89 | 0.24 |
| 6 | 0.8 | 0.7 | 6 | 30 | 66.67 | 8 | 0.83 | 0.22 |
| 7 | 0.9 | 0.8 | 7 | 35 | 57.14 | 5 | 1.14 | 0.31 |
| 10 | 1 | 0.9 | 10 | 50 | 40 | 4 | 1 | 0.27 |
| 12 | 1.1 | 1 | 12 | 60 | 33.33 | 4 | 0.83 | 0.22 |

Table 4 shows optimization of tubular rosette folding of a TC for reducing Implant Area in a human. Tubular-folded TCs can be bundled to afford trocar-implantable devices. A 4-petal rosette affords trocar-implantable bundles with the smallest Implant Area. This is our optimized Origami Macroencapsulation Device.

TABLE 4

| | | | | | human device, 1.4 cm trocar, 500k islets | | | | human device, 1.4 cm trocar, 1 million islets | | | |
|---|---|---|---|---|---|---|---|---|---|---|---|---|
| # of | OD | ID | islets | islets/mm | length/500k | | device length | IA | length/10⁶ | | device length | IA |
| petals | (mm) | (mm) | (in x-section) | length | islets (mm) | #rods** | (cm) | (cm²) | islets | #rods** | (cm) | (cm²) |
| 1 | 0.35 | 0.25 | 1 | 5 | 100000 | 1245 | 8.03 | 11.24 | 200000 | 1245 | 16.06 | 22.49 |
| 2 | 0.5 | 0.4 | 2 | 10 | 50000 | 607 | 8.24 | 11.53 | 100000 | 607 | 16.47 | 23.06 |
| 4 | 0.6 | 0.5 | 4 | 20 | 25000 | 421 | 5.94 | 8.31 | 50000 | 421 | 11.88 | 16.63 |
| 5 | 0.7 | 0.6 | 5 | 25 | 20000 | 307 | 6.51 | 9.12 | 40000 | 307 | 13.03 | 18.24 |
| 6 | 0.8 | 0.7 | 6 | 30 | 16666.67 | 235 | 7.09 | 9.93 | 33333.33 | 235 | 14.18 | 19.86 |
| 7 | 0.9 | 0.8 | 7 | 35 | 14285.71 | 185 | 7.72 | 10.81 | 28571.43 | 185 | 15.44 | 21.62 |
| 10 | 1 | 0.9 | 10 | 50 | 10000 | 149 | 6.71 | 9.40 | 20000 | 149 | 13.42 | 18.79 |
| 12 | 1.1 | 1 | 12 | 60 | 8333.33 | 123 | 6.78 | 9.49 | 16666.67 | 123 | 13.55 | 18.97 |

Through iterations of petal numbers (Tables 3-4), 4 petals was found to be optimal for Implant Area (IA) and geometric packing. Whereas the human-sized TC is surgically-infeasible to implant, a human-sized trocar-implantable tubular rosette can be non-invasively inserted subcutaneously via 1.4 cm trocar in 1-2 ca. 5.94 cm-long devices, depending on the number of islets required to restore euglycemia (500,000-1,000,000). Thus, this single example of TC folding demonstrates the potential to greatly decrease the IA of the TC.

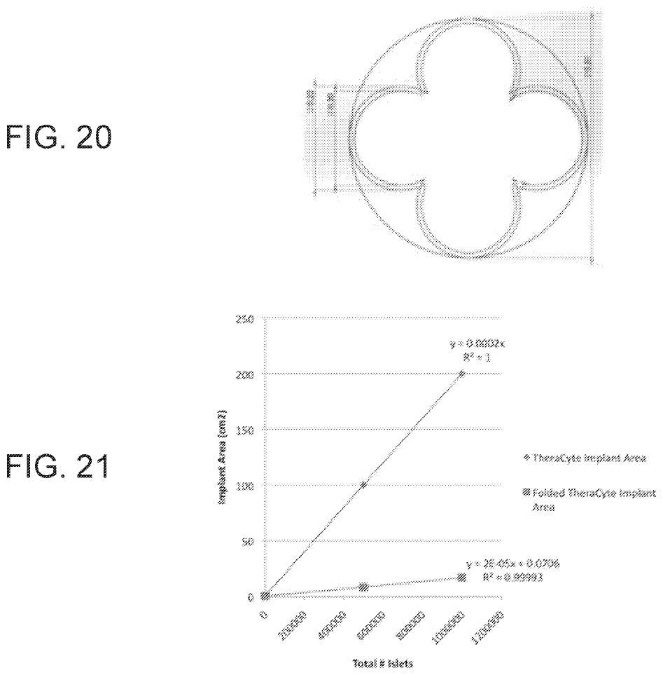

Thus from mathematical modeling of folded TC structures it was determined that Implant Area is an important criteria for determining surgical invasiveness and practicality of a subcutaneous macroencapsulation device; nature (e.g. GI surface area folding) and human art (e.g. origami) can provide examples of folding as a means to preserve surface area while condensing the effective size of objects; and square packing mathematical modeling predicts that a 4-petal tubular rosette (“folded” TC, see FIG. 20) with 8.31-16.63 cm² implant area could effectively treat or cure a human with T1D. Such a device is referred to herein as the Origami Macroencapsulation Device.

Head-to-head mathematical comparison of existing TCs to geometrically-optimized device (Origami Macroencapsulation Device)

Based on mathematical models of the TC and the Origami MED, the relationship between Implant Area (IA) and viable, encapsulated islets is linear for both devices (FIG. 21). Notably, the human-sized Origami MED (projected IA=8.31 cm²-16.63 cm²) has a 91.7% reduction in IA compared to a human-sized TC.

To determine if the Origami MED would be feasible for implantation, we compared its size to that of standard implantable devices. The dimensions of this trocar-implantable device are: diameter=1.4 cm, length 5.94 cm (per 500,000 islets) or 11.88 cm (per 1,000,000 islets).

A 2 mL Alzet osmotic pump is a standard, subcutaneously-implantable device used in animals as small as rats. The dimensions of the Alzet pump are: diameter=1.4 cm, length=5.1 cm. Given that up to two 2 mL pumps may be implanted in the same rat, we conclude that a the similarly-sized Origami MED could not only be implanted in humans, but also could likely be implanted in rodents. Thus, if we could fabricate such a device, human-sized macroencapsulation devices (containing 500,000 to 1,000,000 IEQs) could be rapidly advanced to rat studies. This would be an unprecedented macroencapsulation study in rodents.

Based on a head-to-head comparison of TC vs Origami MED, it was determined that: both TC and Origami MED have linear relationships between implant area and islet loading; the projected human-sized Origami MED reduces the implant area of TC by 91.7%; the projected human-sized Origami MED (for 500,000 islets) is comparable in size to one rat-compatible 2 mL Alzet pump. Because two of these pumps can be implanted in large rats, a rodent study with human-sized devices is feasible; and a rodent-based proof-of-concept study using a human-sized device may allow rapid advances toward clinical trials.

### A Novel Method for Fabricating Geometrically-Optimal Devices: Print-Coating (PC)

Problems with Standard Fabrication Methods

The TC was optimized for fabrication with traditional methods, which depend on 2D membranes to form devices. Due to the delicacy of 2D membranes, deformation of pores when bent, and blockage of pores when over-handled or exposed to adhesives, such devices are constrained to 2D geometries in which the flat shape of the membrane is undisturbed. Thus, a new method is required to fabricate the complex, 3D membrane structures of the geometrically-optimized Origami MED.

### The Print-Coating (PC) Concept

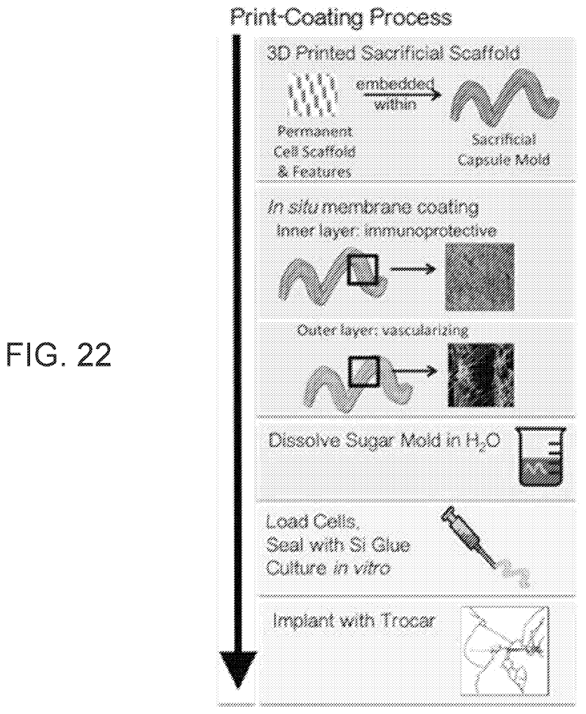

Rather than manipulate pre-constructed commercial membranes, print-coating aims to form precision 3D membrane geometries in situ, thereby generating completed macroencapsulation devices in a single step. The 3D architecture is achieved by 3D-printed scaffolds, onto which membranes are directly deposited. Sacrificial elements of the scaffold are removed from the device lumen to form the completed device (See discussion of Print-Coating Process Schematic, below; see FIG. 22 for a diagram of a print-coating process).

### 3D Printed Scaffolds for Macroencapsulation

Scaffolds for cell culture within the macroencapsulation device are formed by 3D printing. The scaffold contains sacrificial components (which are removed after device completion), with or without non-sacrificial components (that provide functions within the cell chamber).

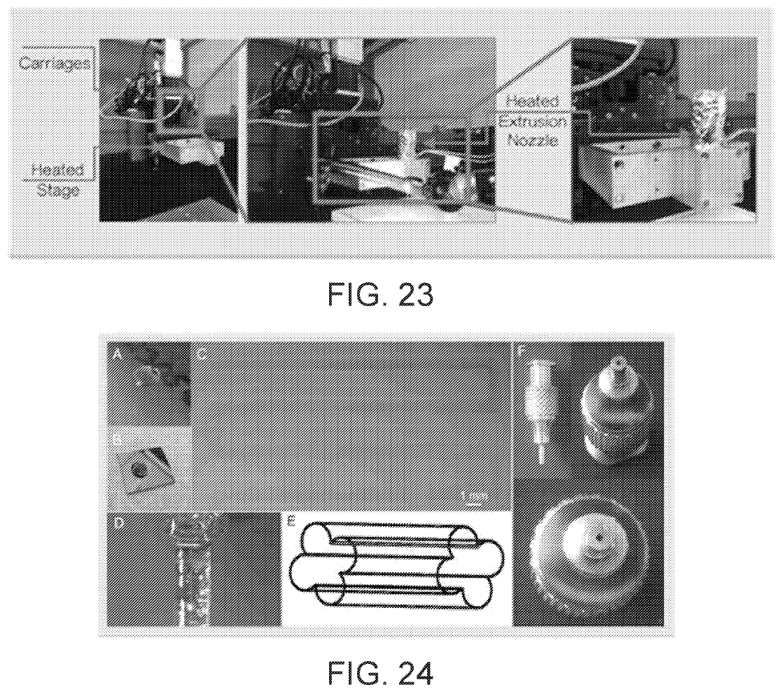

Sacrificial components: This 3D-printed component forms the complex 3D features of the cell capsule, upon which the capsule membranes are deposited. Any material that can be selectively removed without disturbing the capsule membranes or non-sacrificial components is acceptable, however non-toxic materials are preferred to avoid residual toxic residues that may harm encapsulated cells or the implant recipient. In certain embodiments a 3D-printed sugar (e.g. glucose with or without a mixture of fructose and dextran) is used for this function (FIG. 23), which readily dissolves in water and whose residues are not biologically harmful. As an alternative to 3D-printing, sugar glass may also be extruded with a basic pulling mechanism to create scaffolds with only sugar components.

Non-sacrificial components: The 3D-printed capsule may be formed from a combination of sacrificial and non-sacrificial components, with the latter creating structural or functional features. For example, cells may be distributed within the device with the aid of a permanent inner lattice/matrix to prevent cell aggregation. Non-degradable components can also form discrete chambers to form perfusion/flow paths; electrical or mechanical sensors; or reservoirs of nutrients, oxygen (e.g., CaO), drugs, etc. Importantly, ‘non-sacrificial’ in this context means the components are not removed during device fabrication—these components may be either permanent features of the device or features intended to change or deplete over the course of implantation. Thus, non-sacrificial components may include: (a) oxygen-producing substances; (b) reservoirs of nutrients; (c) components to enhance vascularization; (d) components to reduce inflammation; (e) reservoirs of drugs; components for the flow of fluid(s); (g) components for the flow of gas(ses); (h) sensors; (i) electrical components; (g) pumps; or (h) loading or refilling ports.

### In Situ Membrane Coating of Devices

Completed scaffolds (with sacrificial +/−non-sacrificial components) are coated with semipermeable membranes with precise architecture and geometry.

Precision architecture: The geometrically-optimized Origami MED aims to recapitulate the membrane architecture of the TheraCyte, which successfully engenders close vascularization (via aFBR, as discussed herein) while affording allograft immunoprotection. To create this architecture, a bilayer of membranes is directed deposited onto the scaffold surface with a membrane-forming technique, such as electrospinning, rotary-jet spinning, force-pulling, etc. Critically, to recapitulate TC architectures, the inner membrane must have a pore size <0.45-1 μm in diameter (for alloprotection), whereas the outer membrane must have a pore size of 5-10 μm with thin struts (<1-2 μm diameter) to prevent fibrotic tissue deposition in vivo. The two layers are readily formed in situ by nanofiber deposition methods, in which the scaffold is rotated by rotor or mandrel in the deposition path. The immunoprotective layer is formed by one set of deposition parameters, while the vascularizing layer is formed by a distinct set of parameters, where “parameters” may include: nanofiber material, solvent, solution viscosity (concentration), injection rate, applied voltage or force, time of flight (distance to collector), and speed of the rotating scaffold. Completed membranes may be sealed into a “net” architecture by solvent vapor annealing as necessary. Although we focus on membrane deposition in this discussion, coating of the devices may be carried out using other techniques/materials including: (a) 3D-printed membranes; (b) rotary-jet-sprayed materials; (c) electrospun materials; (d) melt-spun materials; (e) hydrogel(s) coating; (f) graphene: (g) metal(s); or (i) inorganic salts.

### Removal of Sacrificial Components

Once membranes are completed, sacrificial components of the scaffold are removed. In the case of sacrificial sugar glass, this material is rapidly dissolved away by soaking devices in water. Following removal, devices are ready for cell loading.

### Cell Loading

The Origami MED may be loaded via a 16G needle through a 16G PTFE loading tube incorporated during the membrane coating process. To achieve high density of packed cells, excess media readily ultrafilters through the capsule membrane. Following loading, the loading tube is trimmed away, and the entry is plugged with silicone glue or another biocompatible epoxy.

### Prior to Implantation

Sealed devices are incubated in the appropriate media (for the cell type encapsulated) at 37° C., 5% CO₂ until implantation.

### Implantation

The Origami MED is designed for subcutaneous trocar injection. However, PC can produce devices of diverse sizes and shapes, and implantation method may be chosen to best suit the device geometry in question. In addition, PC devices are also compatible with prevascularization in vivo prior to loading cells. They may also be implanted with intact loading ports for percutaneous reloading or infusion of other fluids or gasses.

### Device Analysis

Basic PC devices (formed with sacrificial components and membranes only) are histology compatible. Devices may also be assessed intact for glucose-stimulated insulin secretion (GSIS) in vitro and in vivo. Cell extraction from devices is possible, but yields <100% loaded cell density.

### Print-Coating Proof-of-Concept

We have constructed prototype Print-Coated macroencapsulation devices for trocar implantation.

FIG. 24 shows optimization of 3D-printed sugar glass. Panels (A and B) show substrate determination. Molten sugar glass removes cleanly from copper and aluminum substrates (Panels A, C, respectively), but adheres to silicon (Panel B). Panel (C) shows horizontal sugar glass printing. Panel (D) shows vertical extrusion (nozzle visible at top). Panel (E) shows a CAD schematic of 4-petal rosette. Rosettes may be achieved via horizontal printing or vertical extrusion. Panel (F) shows a custom-machined stainless steel nozzle for extrusion of 4-petal rosettes (top left) side view showing Luer lock, (top right) view of nozzle tip, (bottom) close-up of nozzle tip. 1 cm scaffolds may be printed or extruded within seconds.

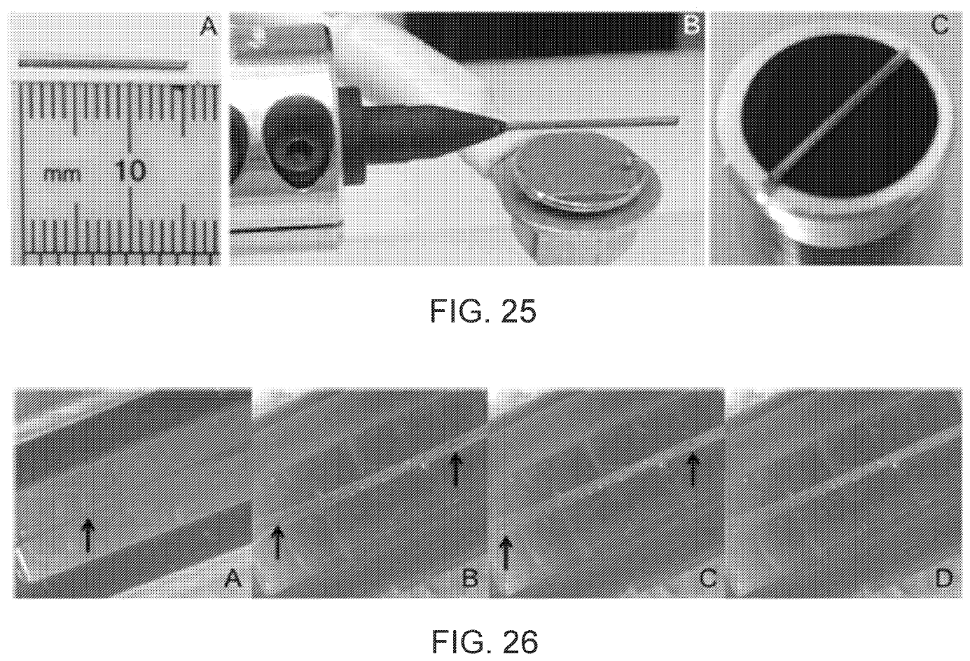

FIG. 25 shows poly-E-caprolactone coating of extruded sacrificial scaffold. Panel (A) shows a sacrificial scaffold (sugar glass, formed by vertical extrusion). Panel (B) shows mounting of scaffold onto rotor (prior to coating). Panel (C) shows a poly-£-caprolactone-coated scaffold (formed by electrospinning). Scaffolds may be coated in bilaminar membranes within minutes.

FIG. 26 shows removal of sacrificial scaffold (sugar glass dissolution in ddH₂O). Panel (A) t=0 min, Panel (B) t=25 see, Panel (C) t=1 min, Panel (D) t=2 min (complete removal).

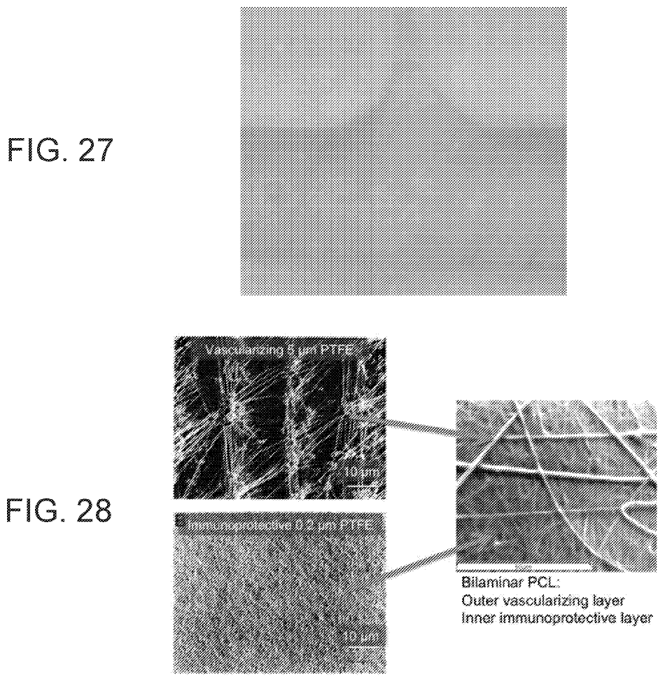

FIG. 27 shows how coated membranes retain sacrificial scaffold architecture. Light microscopy of poly-£-caprolactone membrane formed over sugar glass scaffold with a spike feature (shown after sugar rod removal). The coated membrane retains the shape of the spike. Membrane thickness ca. 45 μm. Membranes were formed by electrospinning. Lumen diameter ca. 500 μm.

FIG. 28 shows how conformal poly-E-caprolactone membrane coating recapitulates TC membrane architecture. Membranes are directly deposited onto the surface of the sacrificial scaffold in two layers to recreate the bilaminar structure of the TC membrane. (Left) SEM of TC membranes. (Right) SEM of analogous, bilaminar PC membrane. (Top) The outer layer promotes vascularization with thin struts and large pore sizes. (Bottom) The inner layer affords alloprotection with small pore sizes. In various embodiments, membranes were formed by electrospinning.

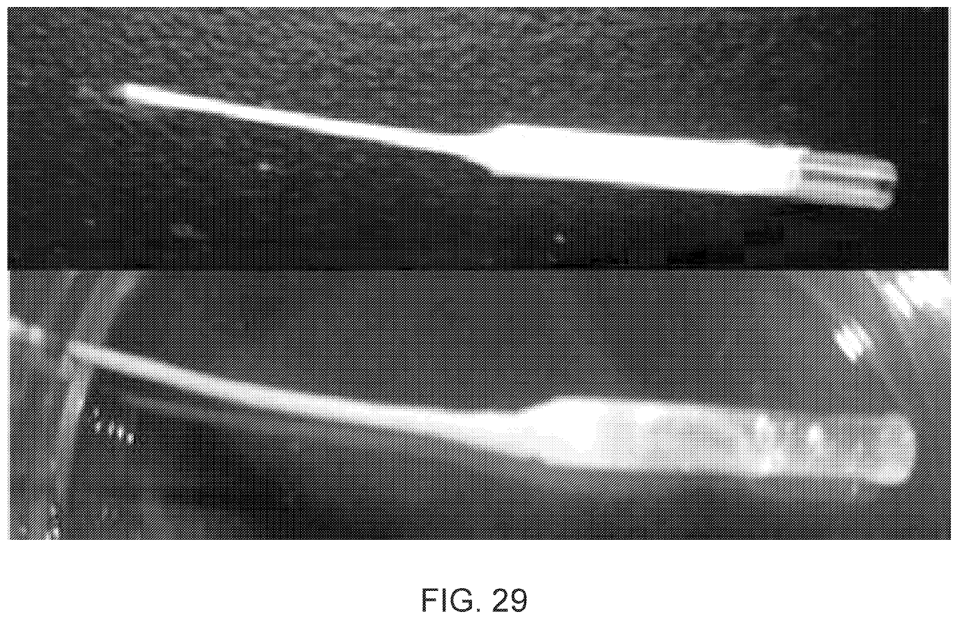

FIG. 29 shows a completed single-rod device. Completed 1.5 cm poly-E-caprolactone device with 16G PTFE loading port, shown prior to sugar scaffold removal (top) and immediately following loading with hESC-derived beta cell clusters (bottom). The loading tube is removed after cells have been packed (not shown). This 1.5 cm-long device accommodates ca. 300 islets. Single-rods may be bundled, increased in length, or both to form scaled-up macroencapsulation devices.

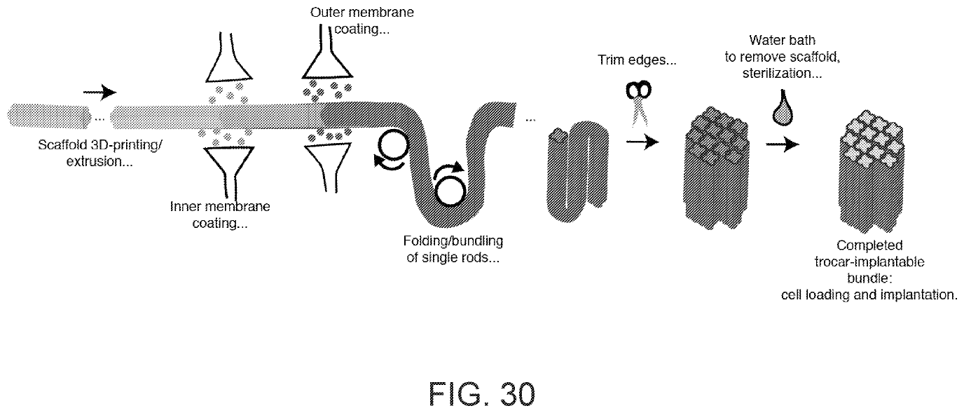

FIG. 30 shows a scaled-up system for mass production. The Print-Coating process is highly scalable for commercial device production. We envision an automated assembly line capable of producing hundreds of completed devices within minutes.

Thus, we have developed a process that allows us to 3D print and coat macroencapsulation devices with intricate microfeatures and geometries. 3D printing of scaffolds affords great versatility in the geometries and features available to macroencapsulation devices. It furthermore allows rapid design, testing, and iteration for rapid advances in prototyping. We have optimized 3D printing and extrusion of sacrificial sugar glass scaffolds to form cylindrical and 4-petal rosette Origami MEDs. In addition, we have recapitulated TC membrane architecture with different materials and coating methods. These initial studies demonstrate the versatility of print-coating as a fabrication strategy for macroencapsulation devices, as well as other membrane-bound devices. Further, we have demonstrated the ability of coated membranes to conform to the geometry of the 3D-printed scaffold. We have proposed a highly scalable system for mass production, such that hundreds of completed devices may be manufactured in an automated assembly line within minutes. Simple devices without non-sacrificial features are cheap and formed entirely of biocompatible materials (sugar, biocompatible polymers, silicone, PTFE).

### Detailed Experimental Justification

As a proof-of-concept for the Origami MED and Print-Coating process, we have developed the following experimental plan:

### In Silica Computational Optimization

We have already presented the mathematical models supporting the optimized geometry of the Origami MED for trocar implantation. We are continuing to explore alternative geometries and strategies for islet packing with computational models and COMSOL simulations, including the incorporation of perfusion and/or 3D-printed cell scaffolds prevent islet aggregation, using Computer-Aided Design and 3D-printing of prototype scaffolds.

### Print-Coated Capsule Materials Evaluation

Completed capsules are assessed across the following metrics for quality and utility prior to in vitro and in vivo study:

Scaffold Geometry: 3D-printed and extruded scaffolds are quality controlled for desired geometries by micrometer, dissecting microscope, and/or SEM. Scaffolds are also visually monitored for quality during the printing or extrusion processes.

Membrane Coating: Parameters for membrane coating of each layer (vascularizing and immunoprotective) are optimized individually by spinning the layers separately and assessing products with SEM. Parameters are thus determined to achieve desired membrane thickness (determined by fiber diameter and duration of deposition), pore size (also determined by fiber diameter and duration of deposition), strut size (i.e., fiber diameter. Controlled by polymer viscosity, molecular weight, solvent, applied force/voltage and time of flight), and uniformity (controlled by scaffold rotation rate and position). Bilaminar membranes are formed by coating with the inner layer protocol, followed by direct coating of the resulting surface with the outer layer protocol.

Integrity: Devices are loaded with test fluid to ensure integrity during ultrafiltration and manipulation.

### In Vitro Assessment of Encapsulated Cell Survival and Function

Number of IEQs to load: Due to heterogeneity in islet size, we must determine the average number of IEQs necessary to densely pack the macroencapsulation device. We thus determine the Packed Cell Volume of IEQs, and from this data estimate the number of IEQs to be loaded to match the internal volume of the device. We assess the validity of this estimate by loading devices with this number of IEQs and assessing packing by histology. The final number of loaded IEQs is adjusted based on these data and used for all subsequent studies.

### In Vitro Survival and Function Assessment:

Histology immediately following loading: Devices are loaded and immediately fixed in 10% formalin, embedded in paraffin, and stained with H&E (to assess morphology and cell health) and insulin immunohistochemistry (to confirm presence of insulin producing cells). We are particularly interested in damage to cells caused by shearing during loading.

Histology after in vitro culture: Devices are loaded and cultured for 4 days before fixation and histology to determine changes in cell morphology, health, and insulin production.

Glucose-stimulated insulin secretion (GSIS): Device function is determined by GSIS challenge on days 1 and 4 of in vitro culture. Whole devices are primed by incubation in basal glucose media for 30 min, washing in PBS, and an additional 30 min incubation in basal glucose media. Devices are rinsed in PBS and transferred to high glucose media, and media samples are collected and every minute for 30 min to measure the concentration of released insulin. Devices are rinsed in PBS and transferred to basal glucose media, and shut-off kinetics are determined by measuring insulin concentration in the surrounding media every minute for 30 min.

### In Vivo Pilot Study

The initial in vivo pilot study is a 1-week implant of prototype Print-Coated devices. Endpoints for the study include: practice loading cells into devices; practice implanting devices; 1-week vascularization assessment; and device integrity and troubleshooting methods.

### In Vivo Survival and Function

Stage 1: Hypoxia during prevascularization period (immunodeficient animal):

Rationale: With no immune onslaught, cell death within implanted capsules (that is not observed within in vitro capsules) will be due to hypoxic stress during the prevascularization period.

Conditions include: 10 SCID beige mice, 1× IEQ-loaded device each, implanted for 2 weeks; 10 SCID beige mice, equivalent number of IEQs injected into kidney capsule; 5 SCID beige mice, 1× TC with equivalent number of IEQs each; and 5 in vitro devices, equivalent number of IEQs cultured during the implant period.

Monitoring during implant period includes monitoring of: in vivo function using glucose tolerance tests (GTTs) on days 1, 4, 7, 10, and 14; and monitoring of in vitro function using GSIS on days 1, 4, 7, 10, and 14.

At implant period end point (day 14), the following data may be collected: 8 explanted devices: ex vivo GSIS, followed by histology (H&E, insulin IHC): 2 explanted devices: directly fixed for histology-FBR and vascularization quantified; 5 TCs directly fixed for histology-control for survival and hypoxia in standard device; and/or in vitro devices: fixed for histology.

Survival and hypoxia assessment: Morphology of cells within explanted devices will be observed by histology. Any necrosis and distance of necrotic layer from capsule surface will be quantified. Explanted histology will be compared to that of TC and in vitro device histology. Death observed within explants but not found within in vitro devices will be attributed to hypoxia during prevascularization. Death will be compared to that of TC devices; if equivalent or better, degree of death will be in target range (goal=match or outperform TC).

Functional assessment: GTTs of animals implanted with devices will be compared to those of animals with kidney capsule implants, including ex vivo GSIS of explants will be compared to the GSIS measurements of the in vitro devices.

Vascularization: Capsule vascularization (in particular, the number of blood vessels within 15 μm of the capsule surface) will be quantified by histological assessment. Milestone: If hypoxic stress does not significantly harm cells within the implanted device (i.e., more so than in TC), we advance to stage 2.

Stage 2: Death by immune onslaught (immunocompetent animal):

Rationale: Stage 1 isolated death by hypoxic stress during pre-vascularization. Stage 2 adds back immune cells to determine if the print-coated device is immunoprotective. Conditions may include: 10 CD1 mice, 1× IEQ-loaded device each, implanted for 2 weeks; and 10 CD1 mice, equivalent number of IEQs injected into kidney capsule.

Monitoring during implant period may include monitoring of: in vivo function using glucose tolerance tests (GTTs) on days 1, 4, 7, 10, and 14; and in vitro function: GSIS on days 1, 4, 7, 10, and 14.

At implant period end point (day 14), the following data may be collected: 8 explanted devices: ex vivo GSIS, followed by histology (H&E, insulin IHC): 2 explanted devices: directly fixed for histology; and/or in vitro devices: fixed for histology.

### Survival Assessment:

Morphology of cells within explanted devices will be observed by histology. Any death will be quantified. Number of infiltrating immune cells (if any) will be quantified. Explanted histology will be compared to that of in vitro device histology. Death observed within explants but not found within in vitro devices nor Stage 1 explants will be attributed to immune onslaught.

### Functional Assessment:

GTTs of animals implanted with devices will be compared to those of animals with kidney capsule implants. In addition, ex vivo GSIS of explants will be compared to the GSIS measurements of the in vitro devices.

### Milestone:

If devices are immunoprotective, we advance to stage 3. If not, smaller pore sizes will be generated by print-coating, and the MWCO of the membranes will be assessed in vitro by agarose bead diffusion test. The validated membranes will be tested by repeating the Stage 2 in vivo experiment.

Stage 3: Death by hypoxic stress of expanding fibrotic capsule (immunocompetent animal):

Rationale: Stages 1 and 2 looked at early timepoints in which death is attributable to hypoxia during prevascularization or immune rejection. Stage 3 extends into timepoints after which fibrosis matures and the thickest fibrotic capsules are observed (3-4 weeks). We hypothesize that our vascularizing membrane will subvert this fibrotic capsule by keeping close vasculature in direct apposition with the capsule membrane.

Conditions may include use of: 10 CD1 mice, 1× IEQ-loaded device each, implanted for 4 weeks; 10 CD1 mice, equivalent number of IEQs injected into kidney capsule.

Monitoring during implant period may include monitoring of: in vivo function using glucose tolerance tests (GTTs) every 3 days, and/or in vitro function using GSIS every 3 days.

At the implant period end point (4 weeks) the following data may be collected: 8 explanted devices: ex vivo GSIS, followed by histology (H&E, insulin IHC); and/or 2 explanted devices: directly fixed for histology.

Foreign body reaction and vascularization assessment:

Explanted capsules will be examined by histology. The thickness of the fibrotic capsule and number of close vessels (within 15 μm of the capsule surface) will be quantified). FBR observations will be correlated with changes in survival and function between Stage 3 and prior stages.

### Survival Assessment:

Morphology of cells within explanted devices will be observed by histology. Any death will be quantified. Explanted histology will be compared to that of in vitro device histology.

### Functional Assessment:

GTTs of animals implanted with devices will be compared to those of animals with kidney capsule implants. In addition, ex vivo GSIS of explants will be compared to the GSIS measurements of the in vitro devices.

Milestone: Most cell death in macroencapsulation occurs due to 3 major challenges during the first month (prevascularization hypoxia, immune onslaught, and FBR hypoxia). If devices maintain the alternative FBR and sustain cells at 4 weeks, this suggests the devices will be able to maintain encapsulated cell survival at long timepoints.

Stage 4: Human-sized device in a rodent-proof-of-concept (immunocompetent animal):

Rationale: The folded macroencapsulation device is compact enough to contain enough cells to treat a human Type 1 Diabetes patient. We wish to demonstrate survival of a human-sized population of cells within a human-sized device. To avoid causing hypoglycemia by high islet burden, we propose to monitor survival of an innocuous cell population. Because 1 IEQ is ca. 1000-2000 cells, we propose to load 5*108-1*109 HEK cells per device.

Conditions may include: 10 CD1 mice, one human-sized device each, implanted for 4 weeks.

### Foreign Body Reaction and Vascularization Assessment:

Explanted capsules will be examined by histology. The thickness of the fibrotic capsule and number of close vessels (within 15 μm of the capsule surface) will be quantified). FBR observations will be correlated with changes in survival and function between Stage 4 and prior stages. Importantly, the infiltration of blood vessels to the surfaces of rods at the inner bundle will be assessed.

### Survival Assessment:

Morphology of cells within explanted devices will be observed by histology. Any death will be quantified and qualitatively assessed by position within the device.

Milestone: We envision this proof-of-concept as a major step toward large animal studies and a device for clinical trials.

### Restoration of Glucose Homeostasis in Diabetic Animals

Rationale: Stages 1-3 of the in vivo survival and function assays show the device is capable of supporting encapsulated cells survival and function. This stage confirms that this survival and function supports glucose homeostasis in rodent models.

Conditions may include: 10 diabetes-induced CD1 mice, 1× IEQ-loaded device each, implanted for 1 year (or until failure); and/or 10 diabetes-induced CD1 mice, equivalent number of IEQs injected into kidney capsule

### Monitoring During Implant Period:

Monitoring may include in vivo function: glucose tolerance tests (GTTs) every week.

### Implant Period End Point (Failure/Death of Animal)

Milestone: Taken together with Stage 4 of the in vivo survival and function assessments, this final experiment provides strong support for advancing the folded macroencapsulation device to large animals (e.g., NHP) and clinical trials.

### CONCLUSIONS

We mathematically demonstrated the geometric limitations of the TC and similar 2D, diffusion-reliant macroencapsulation devices. A TC capable of holding enough cells but compact enough for implantation in humans would treat or cure T1D, as well as a vehicle for cell-based therapies for many diseases. Thus, we sought to geometrically optimize diffusion-reliant macroencapsulation devices to generate appropriately-sized implants. In addition, we mathematically designed and modeled an improved, 3D macroencapsulation geometry that harnesses the power of folding to create condensed TC devices. This Origami MED could hold enough islets to effectively cure adult T1D patients in an implant small enough for testing in rodents. Further, we developed a scalable fabrication method capable of mass producing these devices (“Print-Coating”). We optimized 3D printing/extrusion of sacrificial scaffolds and membrane coating by electrospinning to form implantable capsules. Finally, we successfully loaded primary and hESC-derived islets into prototype capsules and are preparing for in vitro and in vitro studies with these capsules.

We will complete the large-scale in vitro and in vivo evaluations of the prototype Print-Coated Origami MED. Final iterations of prototype design with pilot in vivo study to ensure immunoprotection and capsule integrity. We will complete in vivo studies with finalized, animal-sized devices (5k hESC-derived islet clusters).

### Cell Selection

Cell progenitor sources such as SCB clusters may have better hypoxia—and cryopreservation-tolerance than primary islets (based on ViaCyte's findings). We will assess hypoxia—and cryopreservation-tolerance of SCB clusters vs. primary mouse+rat islets. We are testing both progenitors and primary islets in pilot studies with Origami MEDs, and we aim to advance device optimization based on the most robust cell source we can access.

### Bundling of Origami Rosettes for Rapid Vascularization

We have built a lab-on-a-chip factory for bundling rods with soft support frames to ensure device integrity and rod spacing (FIG. 31). We will use this support system to control rod position and assess in vivo vascularization/survival with different spacing arrangements to find optimal positions. We can enhance devices by using this method to incorporate hydrogels between rods to promote vascularization. Hydrogels may be impregnated with nutrients, oxygen-eluting materials, and/or angiogenic factors to support encapsulated cells during prevascularization.

Mathematical modeling and optimization of the geometry of human-sized Origami MED for trocar implantation has been performed, producing a device which is a cylindrical bundle of rosette-rods, length ca. 4 cm, diameter 1.4 cm (holds 500,000 IEQ). Optimized geometries have been fabricated using Print-Coating (PC). Print-Coated devices successfully fabricated in multiple materials and characterized. We have safely packed healthy islets in Origami MEDs, which includes having determined methods to maintain device integrity during fabrication, ell loading and culture; having developed efficient loading strategies, encapsulating cells without damage by shearing (as determined by light microscopy); and having established GSIS and histology protocols for evaluating encapsulated cell survival and function. We found that islets retain function in Origami MEDs in vitro and SCB clusters produce and secrete insulin in in vitro pilot study (GSIS, insulin IHC); TCs will be included in full scale study for comparison. Finally, we approximated TC membrane architecture with PC membranes (to engender alternative Foreign Body Response, aFRB). SEM images of capsule membranes show appropriate membrane architecture for aFBR. In vivo pilot studies show close vascularization of the capsule within 7 days. Extended in vivo studies are planned to find if aFBR is maintained.

We will demonstrate that initial hypoxic death during prevascularization of this device is less than or equivalent to that of a TC device. In vivo studies of trocar bundles with SCID mice are planned. Further, we will demonstrate that initial hypoxic death in this device is less than or equivalent to that of a TC device, with in vivo studies in immunocompetent mice to follow. We will also demonstrate that hypoxic death by expanding FBR in this device is less than or equivalent to that of a TC device, with long-term in vivo studies with immunocompetent mice and animal-sized Origami MEDs to follow. We will demonstrate that glucose control with this device in diabetic animals is greater than or equivalent to that of a TC device. Finally, we will demonstrate that a human-dose of IEQs implanted in rats maintains survival and function.

### Cell Loading

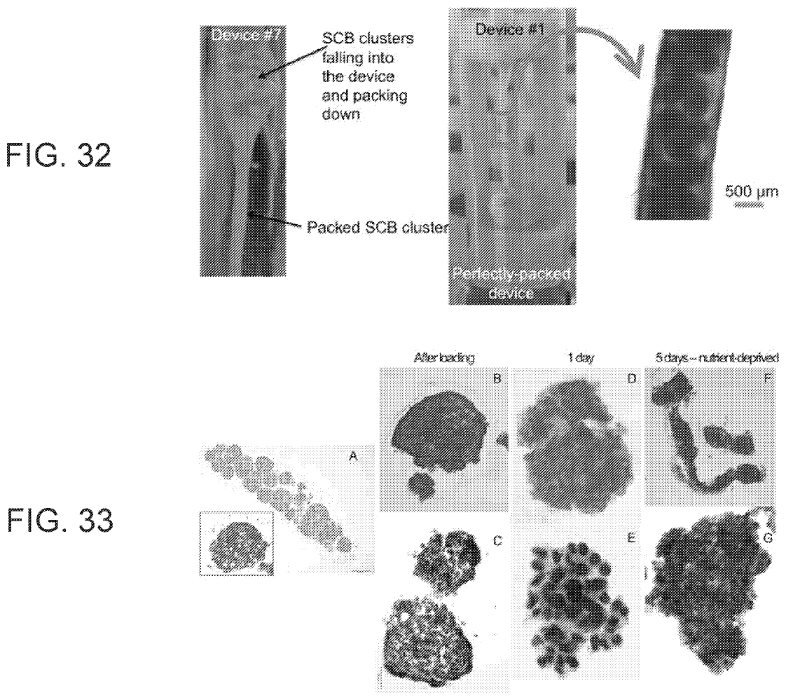

We have produced Print-Coated prototype encapsulation device units and demonstrated the feasibility of loading cells, primary rat islets, and human embryonic stem cell-derived beta cell clusters (SCBs) by a combination of gravity-packing and ultrafiltration. Devices are tightly packed with undetectable damage to cells (FIGS. 32 and 33).

### Cell Survival and Function

We have assessed loaded SCB clusters for survival and function by histology and KCl challenge. Device sections show healthy SCB morphology and insulin production (H&E, insulin DAB—FIG. 33) immediately after loading. This indicates there is minimal damage to SCBs by the loading process. SCBs maintain healthy morphology and insulin production after 1 day in culture, as well as 5 days after nutrient-deprivation challenge. Encapsulated cells produce insulin detectable in the culture media.

### Hydrogel Scaffolding

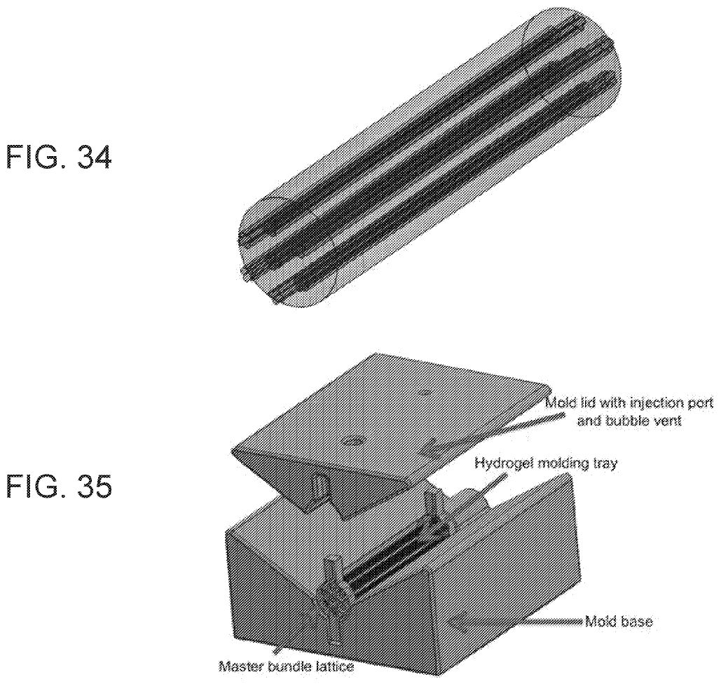

One possible embodiment of the encapsulation device includes a hydrogel support system to maintain folded structures in the correct geometries. Furthermore, this hydrogel may be impregnated with some combination of vasculogenic molecules (e.g., VEGF), oxygen-eluting salts, anti-inflammatory agents or other drugs, and essential nutrients to support encapsulated cell viability during the pre-vascularization period and to promote angiogenesis into the folds of the device (FIG. 34). We have developed a bundling factory to mold device units into the desired folded geometry and to fix this geometry in place (FIGS. 31 and 35).

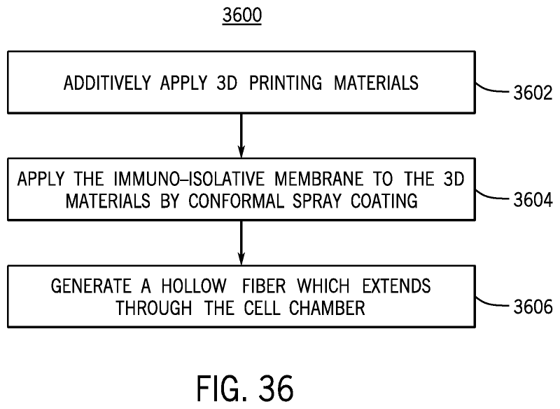

FIG. 36 shows an example 3600 of a process for fabricating a cell encapsulating implantable device in accordance with some embodiments of the disclosed subject matter. The device may include a cell chamber accommodating a plurality of biological cells disposed within a fluid, the cell chamber at least partially enclosed within an immuno-isolative membrane which permits diffusive exchange of nutrients between the fluid and a tissue in which the device is implanted for sustaining the plurality of biological cells. As shown in FIG. 36, at 3602, process 3600 can additively apply 3D printing materials. At 3604, process 3600 can apply the immuno-isolative membrane to the 3D materials by conformal spray coating. Finally, at 3606 process 3600 can generate a hollow fiber which extends through the cell chamber. The hollow fiber may include a semi-permeable surface in communication with the plurality of biological cells.

It should be understood that the above described steps of the process of FIG. 36 can be executed or performed in any order or sequence not limited to the order and sequence shown and described in the figures. Also, some of the above steps of the processes of FIG. 36 can be executed or performed substantially simultaneously where appropriate or in parallel to reduce latency and processing times.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A cell encapsulating implantable device, comprising:
a cell chamber accommodating a plurality of biological cells disposed within a fluid, the cell chamber at least partially enclosed within an immuno-isolative membrane which permits diffusive exchange of nutrients between the fluid and a tissue in which the device is implanted for sustaining the plurality of biological cells, and
the cell chamber being configured to accommodate flow of fluid therethrough, and/or the cell chamber being provided as a condensed 3D shape.

2. The device of claim 1, further comprising a pump for inducing flow of the fluid within the cell chamber.

3. The device of claim 2, wherein the pump induces convective flow within the cell chamber, wherein the device further comprises a conduit fluidly connected to the cell chamber, wherein the pump is coupled to the cell chamber via the conduit, and/or wherein the immune-isolative membrane at least partially enclosing the cell chamber comprises a semi-permeable material configured to prohibit transfer of immunocompetent cells or immunoglobin molecules.

4-5. (canceled)

6. The device of claim 3, further comprising a hollow fiber fluidly coupled to the pump and the cell chamber,
wherein the hollow fiber comprises a semi-permeable membrane.

7. The device of claim 6, wherein the hollow fiber extends through the cell chamber.

8. The device of claim 7, wherein the semi-permeable membrane of the hollow fiber permits diffusive exchange of nutrients between the fluid and the tissue and prohibits transfer of immunocompetent cells or immunoglobin molecules between the fluid and the tissue.

9. The device of claim 8, wherein the semi-permeable membrane of the hollow fiber has a 100 kDa molecular weight cut-off.

10. The device of claim 9, wherein the pump delivers the fluid to the cell chamber in a unidirectional flow path, and wherein the cell chamber comprises an efferent perfusate outlet port-; and/or the pump and the cell chamber are fluidly coupled in a closed loop that recirculates the fluid; and/or the hollow fiber comprises an alternate foreign body response (aFBR)-promoting membrane; and/or wherein the device further comprises at least one equilibrium chamber adjacent to the cell chamber, wherein the hollow fiber extends through the at least equilibration chamber; and/or wherein the hollow fiber comprises a rosette-shaped cross-section.

11-16. (canceled)

17. The device of claim 1, wherein
i. the cell chamber is further defined by a wall, and wherein the wall comprises, embedded in the device in fluid communication with the fluid, at least one of an oxygen-producing substance, a vascularization enhancing component, an inflammation-reducing component, a growth factor, a drug reservoir, a chemical sensor, or an electrical component; and/or
ii. wherein the device has a cross-sectional diameter less than 2 cm and a length less than 5 cm; and/or
iii. wherein the cell chamber is fabricated by deposition of 3D printing materials.

18-19. (canceled)

20. The device of claim 17, wherein the 3D printing materials include sacrificial and non-sacrificial components, and/or wherein the plurality of biological cells are filled into vacant spaces in the cell chamber which are produced by removal of the sacrificial components.

21. (canceled)

22. The device of claim 20, wherein a semi-permeable membrane is applied to the 3D printing materials by conformal spray coating.

23. The device of claim 22, wherein the semi-permeable membranes comprises at least one material selected from: PTFE, a 3D-printed material, a rotary-jet-sprayed material, an electrospun material, a hydrogel, a graphene, a metal, or an inorganic salt.

24. The device of claim 1, wherein the plurality of biological cells comprises a plurality of pancreatic islet cells.

25. The device of claim 24, wherein the plurality of pancreatic islet cells are present at a density of at least 2.5 IEQ/μL-; and/or wherein the plurality of biological cells are embedded within the cell chamber in a hydrogel.

26. The device of claim 25, wherein the plurality of pancreatic islet cells comprises a sufficient number of insulin-secreting islet cells to treat type-1 diabetes (TID).

27. (canceled)

28. The device of claim 1, further comprising a hollow fiber fluidly coupled to the cell chamber,
wherein the hollow fiber comprises a semi-permeable membrane.

29. The device of claim 28, wherein the hollow fiber is provided as a condensed 3D shape, such as wherein the condensed 3D shape comprises at least one of a rosette, a woven structure, a braid, a helix, a spiral, a sinusoid, a cylinder, a rectangular prism, a semi-sphere, a dome, a tube, an ellipsoid, or a multi-pointed star.

30. (canceled)

31. The device of claim 1, further comprising a conduit traversing the device which includes an inner portion containing a vascularizing feature promoting blood vessel growth into the device.

32. The device of claim 2, wherein the pump is implantable or percutaneous,
wherein the pump operates at least one of continuously, temporarily, intermittently or in a pulsatile mode, and
wherein the pump comprises at least one of an infusion pump, an insulin pump, a refillable pump, a micro piezoelectric pump, or a modified microperistaltic pump.

33. A method of fabricating a cell encapsulating implantable device, the device including:
a cell chamber accommodating a plurality of biological cells disposed within a fluid,
the cell chamber at least partially enclosed within an immuno-isolative membrane which permits diffusive exchange of nutrients between the fluid and a tissue in which the device is implanted for sustaining the plurality of biological cells,
the method comprising:
additively applying 3D printing materials;
applying the immuno-isolative membrane to the 3D materials by conformal spray coating; and
generating a hollow fiber which extends through the cell chamber, the hollow fiber comprising a semi-permeable surface in communication with the plurality of biological cells.

34-63. (canceled)
